SEQUENCE LISTING (3 sequences)

SEQ ID NO: 1 - protein, 652 aa, synthetic construct
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 2 - protein, 713 aa, Campylobacter jejuni
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 3 - protein, 19 aa, unidentified
MKKIWLALAGLVLAFSASA

(19) United States
(12) Patent Application Publication
(10) Pub. No.: US 2026/0199454 A1
(43) Pub. Date: Jul. 16, 2026

(54) BIOCONJUGATE COMPOSITIONS AND PROCESS FOR BIOCONJUGATE PRODUCTION

(71) Applicant: Janssen Pharmaceuticals, Inc., Titusville, NJ (US)

(72) Inventors: Helen Heide, Erika THOMAS, Bern (CH); Marc Sylvain NOVERRAZ, Bern (CH); Denis Louis Raymond BASCOUL, Bern (CH); Katharina Christin BAUER, Bern (CH); Eric Nicolaas KLIJN, Leiden (NL); Piergiuseppe NESTOLA, Bern (CH); Leonor MONTEIRO TEIXEIRA PRATA, Bern (CH); Martin Maximilian SCHEIDT, Bern (CH); Renzo DANUSER, Bern (CH); Caspar Ludwig Hermann SCHÄFER, Bern (CH)

(21) Appl. No.: 19/135,521

(22) PCT Filed: Dec. 4, 2023
(86) PCT No.: PCT/US2023/082285

§ 371 Date: Jun. 4, 2025

(30) Foreign Application Priority Data
Dec. 5, 2022 (EP) 22 211 401.9

(51) Int. Cl. A61K 39/385; A61K 39/00; A61K 39/108; C12P 19/28; C12P 21/00; C12R 1/19

(52) U.S. Cl. CPC A61K 39/385; A61K 39/0258; C12P 19/28; C12P 21/005; A61K 2039/6037; C12R 2001/19

(57) ABSTRACT

The present invention relates to improved pharmaceutical compositions comprising one or more bioconjugate compositions. Each bioconjugate composition comprises a mixture of mono-glycosylated and multi-glycosylated bioconjugates of specific E. coli O-antigen polysaccharide covalently coupled to a Pseudomonas aeruginosa Exoprotein A carrier protein. The invention further relates to a process for the production of purified bioconjugates from Gram-negative host cells and to bioconjugates obtained or obtainable by said process.

### CROSS REFERENCE TO RELATED APPLICATION

The present application claims priority to European patent application EP 22 211 401.9, filed Dec. 5, 2022, the disclosure of which is incorporated herein by reference in its entirety.

### REFERENCE TO SEQUENCE LISTING SUBMITTED ELECTRONICALLY

The contents of the electronic sequence listing (CRU6090EPEPA1.xml; Size: 4,785 bytes; and Date of Creation: Nov. 28, 2023) is herein incorporated by reference in its entirety.

### BACKGROUND OF THE INVENTION

In contrast to commensal E. coli, extraintestinal pathogenic Escherichia coli (ExPEC) strains express a broad array of virulence factors enabling them to colonize the gastro-intestinal tract, as well as to cause a wide range of extraintestinal infections, which are associated with a significant healthcare cost burden due to hospitalization and death. Neonates, the elderly, and immunocompromised patients are particularly susceptible to ExPEC infection, including invasive ExPEC disease (IED).

The O-antigen comprises the immunodominant component of the cell wall lipopoly-saccharide (LPS) in Gram-negative bacteria, including E. coli. There are currently >180 serologically unique E. coli O-antigens identified, with the vast majority of ExPEC isolates classified within less than 20 O-antigen serotypes. Full-length E. coli O-antigen polysaccharides (O-PS) are typically comprised of about 10 to 25 repeating sugar units. In wild type E. coli, such O-PS are attached to the highly conserved LPS core structure. Each of the aforementioned components, i.e. O-PS component and LPS core structure, are synthesized separately by enzymes encoded predominantly in the rfb and rfa gene clusters, respectively. Following polymerization of the O-antigen, the O-PS backbone may be modified, typically through the addition of acetyl or glucose residues.

Efforts toward the development of a vaccine to prevent ExPEC infections have focused on multivalent compositions comprising bioconjugates of E. coli O-antigen polysac-charide covalently attached to a carrier protein [see e.g. Poolman and Wacker, J. Infect. Dis. (2016) v.213 (1), pp. 6-13; WO 2015/124769; WO 2017/035181; WO2020/191082A1].

In general, it has been shown that efficient activation of CD4⁺ T-cells by glycoconjugate vaccines is driven by the endosomal generation of antigenic fragments of the polysaccharide that are covalently linked to peptides originating from the carrier protein [see e.g. Avci et al., Nat. Med. (2011), 17:1602-1609].

While representing a promising class of vaccines, the production of complex bioconjugates in general, and, in particular the production of bioconjugates comprising a bacterial O-antigen polysaccharide covalently coupled to a carrier protein, is a challenging task. However, several methods of production, including the purification of such bioconjugates have been described [see e.g. WO2009/104074; WO2020/191082; WO 2022/214620; van den Dobbelsteen et al., Vacine (2016), 34:4152-4160].

In general, O-EPA bioconjugates can be produced by enzymatic conjugation of the O-antigen polysaccharide (O-PS) component to a carrier protein in Gram-negative bacterial host cells, such as E. coli, using e.g. the PglB oligosaccharyltransferase system [see e.g. WO 2015/124769; WO 2020/191082; Poolman and Wacker, J. Infect. Dis. (2016) v.213 (1), pp. 6-13 and references therein]. In this method, coupling of the O-PS component to the carrier protein occurs in the periplasmic space, i.e. the space between the inner cytoplasmic membrane and the outer membrane of Gram-negative bacteria, such as E. coli.

Specifically. WO 2022/214620 describes a process for O-EPA bioconjugate production at commercial scale. This process thus represents a significant improvement over previous processes for O-EPA bioconjugate production that included a size exclusion chromatography step (SEC), which rendered these processes less favourable for large scale manufacturing [e.g. van den Dobbelsteen et al., Vaccine (2016), 34:4152-4160; Burckhardt et al., Vaccine (2019), 37 (38): 5762-5769; Ravenscroft et al., Glycobiology (2016), 26 (1): 51-62; WO 2009/104074; or WO 2015/124769]. Thus WO 2022/214620 described for the first time a process that is suitable for the large scale production of O-EPA bioconjugates, such as production in bioreactors with a volume of 100 L up to 20,000 L. However, despite its significant advantage compared to previous methods, this process may be further improved, for example, in order to provide bioconjugate compositions with a relatively higher proportion of multi-glycosylated forms of O-EPA bioconjugates, i.e. EPA carrier protein in which more than one glycosylation site is occupied, e.g. EPA carrier protein attached to 2, 3 or 4 O-antigen polysaccharides. In turn, a higher proportion of multi-glycosylated forms of O-EPA bioconjugates results in a lower amount of carrier-protein relative to the amount of O-antigen polysaccharide. This is of particular relevance as a minimized amount of carrier protein mitigates a hypothetical risk of adverse events induced by the carrier protein or carrier-protein induced immune suppression [see e.g. Juergens et al., Hum Vaccin Immunother. 2018; 14 (8): 1948-1956; Knuf et al. Vaccine 2011; 29 (31): 4881-4890].

Despite generally being well tolerated, it is important to develop pharmaceutical compositions with minimized risk for adverse events, even if the risk is hypothetical. This is particularly relevant for the development of vaccines, which are typically administered to healthy people.

In view of the above described mechanism of T-cell action (Avci et al., Nat. Med. (2011), 17:1602-1609) and the goal to develop vaccines with a minimized risk of adverse events, the development of bioconjugate compositions with a relatively higher degree of multiglycosylated forms and a method for producing such bioconjugates is considered a major improvement over currently known bioconjugate compositions and production methods.

Thus, there is an unmet medical need to provide improved O-EPA bioconjugate compositions and, correspondingly, improved pharmaceutical compositions comprising one or more O-EP A bioconjugate compositions.

Furthermore, those of skill in the art understand that an efficient manufacturing process is needed to obtain sufficient amounts of safe product for vaccination of a large number of people in an economically feasible manner. Thus, there is an ever-existing need for improved processes to manufacture and/or purify such complex bioconjugates. For example, besides improvements related to certain attributes of the thereby obtained bioconjugates per se, e.g. an improved degree of glycosylation, the process described in WO 2022/214620 may be further improved with respect to efficient large-scale manufacturing of the bioconjugates (e.g. culturing of the host cell culture in bioreactors with a volume of at least 100 L up to 20,000 L). Such improvements may inter alia relate to yield, purity and buffer consumption. For example, a reduced buffer consumption is desirable for both, economic (lower costs) and environmental reasons (less waste).

In consequence, a need remains for an improved process for the production of a purified bioconjugate comprising a bacterial O-antigen polysaccharide covalently coupled to a carrier protein, in particular E. coli O-antigen-EPA bioconjugates, addressing the above shortcomings.

### SUMMARY OF THE INVENTION

The present invention relates to improved pharmaceutical compositions comprising one or more bioconjugate compositions. Each bioconjugate composition comprises a mixture of bioconjugates, each bioconjugate comprising bacterial O-antigen polysac-charide of a specific E. coli serotype covalently coupled to a Pseudomonas aeruginosa Exoprotein A carrier protein (EPA; O-EPA bioconjugate). Each O-EPA bioconjugate may be either mono-glycosylated with 1 O-antigen polysaccharide attached to EPA or multi-glycosylated with 2, 3 or 4 O-antigen polysaccharides attached to EPA, so that the bioconjugate composition is a mixture of mono-glycosylated and multi-glycosylated forms of the O-EPA bioconjugate. In certain embodiments, the EP A carrier protein has the amino acid sequence of SEQ ID NO: 1. The invention further relates to a process for the production of purified O-EPA bioconjugates from Gram-negative host cells. The process comprises providing the bioconjugate in a filtered periplasmic fraction (FPF) of the host cells and a purification of said bioconjugate. The purification comprises the steps of (i) a first anion exchange chromatography prior to (ii) a mixed-mode chromatography (MMC) step, followed by (iii) a hydrophobic interaction chromatography (HIC) and (iv) a second anion exchange chromatography. The invention further relates to O-EPA bioconjugate compositions obtainable or obtained by a method according to the invention.

### DESCRIPTION OF THE INVENTION

The above objectives are achieved by a pharmaceutical composition as defined in claim 1, a process for the production of O-EPA bioconjugates as defined in claim 5 and the O-EPA bioconjugate compositions obtained or obtainable by said process as defined in claim 16. Further aspects of the invention are disclosed in the specification and inde-pendent claims, preferred embodiments are disclosed in the specification and the dependent claims.

The present invention will be described in more detail below. It is understood that the various embodiments, preferences and ranges as provided/disclosed in this specification may be combined at will. Further, depending on the specific embodiment, selected definitions, embodiments or ranges may not apply.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the terms “including”, “containing” and “comprising” are used herein in their open, non-limiting sense. It is understood that the various embodiments, preferences and ranges may be combined at will.

As used herein, the term “about,” when used in conjunction with a number, refers to any number within +10%, preferably +5% or +1% of the referenced number.

Throughout this specification a number of abbreviations are used, including:
AEX1 a first Anion EXchange chromatography
AEX2 a second Anion EXchange chromatography
cGE Capillary Gel Electrophoresis
CHA ceramic hydroxyapatite
CPF centrifuged periplasmic fraction
CV column volume
DOGY degree of glycosylation
EPA ExoProtein A (also referred to as exotoxin A) of Pseudomonas aeruginosa
ExPEC extra-intestinal pathogenic E. coli
FPF Filtered Periplasmic host cell Fraction
HA Hydroxyapatite
HCP Host Cell Proteins
HIC Hydrophobic Interaction Chromatography
HPLC High-Performance Liquid Chromatography
IC-PAD Ion chromatography with pulsed amperometric detection
IED invasive ExPEC disease
MMC mixed-mode chromatography
MMR multimodal chromatography resin
O-EPA bioconjugate of EPA carrier protein covalently coupled to bacterial O-antigen polysaccharide
PF periplasmic fraction
RP-HPLC Reversed Phase-High-Performance Liquid Chromatography
SEC Size Exclusion Chromatography
SE-HPLC Size Exclusion-High-Performance Liquid Chromatography
SEPA short-chain glycosylated EPA
TFF Tangential Flow Filtration
WFI water for injection

The term “glycoconjugate” is known in the field and particularly describes chemical entities covalently bound to one or more polysaccharide(s). Such glycoconjugate may be obtained by biological conjugation in a living cell (“bioconjugate” or “biological conjugate”) or may be obtained by chemical conjugation of a polysaccharide (“chemical” or “synthetic” glycoconjugate). Particularly suitable chemical entities are proteins, the corresponding glycoconjugates being glycoproteins. Specifically, the term glycoconjugates relates to a conjugation product wherein a polysaccharide (i.e. a glycan) is covalently coupled to a carrier protein. If the carrier protein comprises more than one glycosylation sites, mixtures of glycoconjugates, particularly bioconjugates, are obtained, e.g. mixtures of mono-, di-, tri-, and tetra-glycosylated bioconjugates. An example of a carrier protein comprising 4 glycosylation site is an EPA carrier protein having an amino acid sequence of SEQ ID NO: 1.

“Bioconjugate compositions” comprise such mixtures, and these may include bioconjugates of one polysaccharide (e.g. as in drug substances of a glycoconjugate of a carrier protein coupled to O-antigen polysaccharide of a specific bacterial serotype; sometimes referred to as “monovalent composition”) or alternatively of more than one polysaccharide (e.g. as in a drug product wherein O-antigen polysaccharides of multiple bacterial serotypes each individually coupled to carrier proteins are included in the same composition; sometimes referred to as “multivalent composition”).

The term glycoprotein includes “glycoconjugate vaccines”. In glycoconjugate vaccines, the emphasis is on the glycan part, to which an immune response is desired because the glycans are the relevant antigens, and the protein part merely serves as a carrier to lead to a desired T-cell memory immune response.

The term “polysaccharide” is known in the field and particularly describes polymeric carbohydrates composed of monosaccharide units bound together by glycosidic link-ages, either linear or branched. Such polysaccharides are characterized by their repeating units, each repeating unit described with their respective monosaccharide composition. Said repeating units include one or more monosaccharides which can also be chemically modified (e.g. amidated, sulphonated, acetylated, phosphorylated, etc). Typically found monosaccharides in said repeating units are cyclic or linear monosaccharides containing three to seven carbon atoms. In the specific case of glycoconjugate vaccines, the conjugated polysaccharide originates from a pathogenic species (e.g. Escherichia coli) with said repeating unit defined by the genetics of the specific pathogen. The repeating unit can thus be a specific marker/identifier of the pathogen.

The term “polysaccharide component” consequently denotes one or more glycan chain(s) of a glycoconjugate. Glycans can be monomers or polymers of sugar residues, but typically contain at least three sugars, and can be linear or branched. A glycan may include natural sugar residues (e.g., glucose, N-acetylglucosamine, N-acetyl neu-raminic acid, galactose, mannose, fucose, arabinose, ribose, xylose, etc.) and/or modified sugars (e.g., 2′-fluororibose, 2′-deoxyribose, phosphomannose, 6′-sulfo N-acetylglucosamine, etc). The term “glycan” includes homo- and heteropolymers of sugar residues. The term “glycan” also encompasses a glycan component of a glycoconjugate (e.g., of a glycoprotein, glycopeptide).

The term “O-acetylated polysaccharide”, as used herein, refers to polysaccharides where one or more monosaccharides of the repeating unit are modified by acetylation. Said monosaccharides have one or more of their present hydroxyl groups acetylated. For pathogen-derived repeating units used in glycoconjugate vaccines, the O-acetylation of certain monosaccharides can influence induction of an immune response for said pathogen. Examples of pathogen-derived polysaccharide components are shown in Table 1.

The terms “glycan”/“glycan chain” are synonyms of “polysaccharide” as defined below. Correspondingly, in the context of this invention, “glycan” and the prefix “glyco-”, also refers to the carbohydrate portion of a glycoconjugate, such as a glycoprotein. Accordingly, “glycosylated” refers to one or more polysaccharides covalently attached to a protein, thus the protein being “monoglycosylated” or “multiglycosylated”, respectively.

The term “serotype” as used herein, refers to glycoconjugates having different polysaccharide chains which are derived from different bacterial serotypes. Examples of glycans from a number of E. coli serotypes are identified below in Table 1.

The term “adjusting a load” as used herein, refers to adjusting a load of a process intermediate, e.g. the periplasmic fraction of a host cell comprising an O-EPA conjugate or the O-EPA comprising fraction after a first, second or third purification step, to conditions that are suitable for applying said process intermediate on a chromatography resin for further purification. Unless specified otherwise or clearly contradicted by the context, “adjusting a load” refers to adjusting the conductivity of a load to a target conductivity that is suitable for the following purification step and/or adjusting the pH of a load to a target pH that is suitable for the following purification step. Further, “adjusting a load” includes adjusting the concentration of a process intermediate, i.e. reducing the processing volume, particularly by TFF.

The terms “resin” and “medium” are used herein synonymously and relate to a chromatography resin or a chromatography medium that is used for separation of a target protein(s), i.e. the O-EPA bioconjugate, from impurities. Resins useful for the invention can be in different formats, e.g. as beads, filters (membranes), cartridges, etc., all to be considered as ‘resin’ according to the invention. In certain embodiments the resins are in the form of beads that can be used in columns. In certain embodiments the resins are in the form of membranes with functional groups. In certain embodiments the resins are in the form of directly usable cartridges. Resins that can be used according to the invention can be commercially obtained from vendors, e.g. Cytiva (former GE Healthcare), Bio-Rad, and/or others.

The term “capture step” is known in the field and relates to a first chromatography step with the objective to bind the protein(s) of interest from the crude sample and to isolate them from critical contaminants such as proteases and glycosidases. The target protein(s), e.g. the O-EPA bioconjugate(s), are concentrated and transferred to a buffer that will maintain the functional and structural integrity of the O-EPA bioconjugate. Removal of other critical contaminants may also be achieved by careful optimization of binding conditions.

The focus in optimizing a capture step is on capacity and speed. It may thus be acceptable to compromise on resolution in order to maximize the capacity and/or speed of the separation in this first step.

The term “polishing step” is known in the field and relates to a chromatography step that is performed as final chromatography step with the objective to further enhance the purity of the target protein, e.g. O-EPA bioconjugate.

The term “bind-elute mode” is known in the field and relates to a mode of separation that works by first binding sample components, in particular the protein/bioconjugate of interest, to the chromatography resin. Once sample components are bound, the resin is washed with a buffer, and thus non-bound material is removed. Then, the bound material is eluted. This mode of separation is in contrast to the flow-through mode wherein the pH/ionic strength of the sample and buffer is selected in such a way that the protein will not bind but will flow through the column, leaving most or specific impurities bound.

In embodiments of the invention, in steps ii) to v) conditions are first adjusted to allow binding of the O-EPA bioconjugate to a chromatography medium and subsequently adjusted to allow elution of the O-EPA bioconjugate from said medium, i.e. the respective chromatography step is performed in bind-elute mode.

Viable and non-viable particles: Viable particles are particles that contain one or more living microorganisms, such as bacteria. These can affect the sterility of the pharmaceutical product.

Non-viable particles are particles that do not contain living microorganisms but may act as transportation vehicles for viable particles.

Viable and non-viable particles generally range from about 0.2 μm to 30 μm in size, typically from about 0.2 μm to 5 μm.

The term “drug substance” as used herein refers to the bulk product of an individual bioconjugate (e.g. E. coli O-antigen polysaccharide covalently coupled to EPA carrier protein, e.g. E. coli O25B O-antigen covalently coupled to EPA), that is at higher concentration than the product as will be finally administered to a subject in need thereof. The drug substance can be produced after purification of the bioconjugate. The drug substance can, for example, be stored in a more concentrated form in a suitable formulation buffer (see e.g. WO 2018/077853, WO2020/191082), for instance in frozen condition, e.g. at minus 70° C.

The term “drug product” as used herein refers to the formulation of the bioconjugates particularly E. coli O-antigen polysaccharides individually coupled to EPA carrier protein, in final form for administration to a subject in need thereof. As used herein, the term “drug product” particularly relates to a multivalent vaccine composition, e.g. a four-valent ExPEC glycoconjugate vaccine composition comprising the E. coli O-antigen polysaccharides O25B, O1A, O2 and O6A, each individually coupled to an EPA carrier protein. Further non-limiting examples of multivalent glycoconjugate vaccine compositions are e.g. a nine-valent glycoconjugate vaccine compositions comprising the E. coli O-antigen polysaccharides O1A, O2, O4, O6A, O15, O16, O18A, O25B and O75, and e.g. a ten-valent glycoconjugate vaccine composition comprising the E. coli O-antigen polysaccharides O1A, O2, O4, O6A, O8, O15, O16, O18A, O25B and O75, each E. coli O-antigen polysaccharide being individually coupled to an EPA carrier protein (see e.g. WO2020/191082 and WO2022/058945 for examples of ten- and nine-valent glycoconjugate vaccine compositions, respectively). Drug product can typically be prepared by mixing the drug substances of the respective glycoconjugates, and dilution by a suitable formulation buffer if needed (see e.g. WO2018/077853, WO2020/191082, WO2022/058945), such that the target dose of vaccine is produced.

In a first aspect, the invention relates to a process for production of a purified bioconjugate from Gram-negative bacterial host cells. Said bioconjugate particularly comprises a bacterial O-antigen polysaccharide covalently coupled to a carrier protein, said carrier protein being Pseudomonas aeruginosa ExoProtein A (O-EPA bioconjugate). It is to be understood that the Pseudomonas aeruginosa ExoProtein A (EPA) carrier protein is a detoxified or recombinant version thereof.

The process comprises providing a filtered periplasmic fraction (FPF) of the host cells that express the bioconjugate (step i). Said FPF comprises the bioconjugate. The process further comprises several chromatographic purification steps (herein described as steps ii to v).

Step ii comprises “subjecting an optionally adjusted load of the FPF to a first anion exchange chromatography (AEX 1) step to obtain a first AEX eluate (AEX1) that comprises the bioconjugate”. This step is a capture-step.

Step iii comprises “subjecting an optionally adjusted load of the AEX1 eluate to a mixed-mode chromatography (MMC) step on a multimodal resin (MMR) to obtain an MMR eluate, said MMR comprising both anion exchange functionalities and hydrophobic functionalities to obtain an MMR eluate comprising the bioconjugate”. Step iv comprises “subjecting an adjusted load of the MMR eluate to a hydrophobic interaction chromatography (HIC) step to obtain a HIC eluate comprising the bioconjugate”.

Step v comprises “subjecting an adjusted load of the HIC eluate to a second anion exchange chromatography (AEX 2) step to obtain an AEX2 eluate comprising the bioconjugate as product”. This step is a polishing step.

In purification steps ii to v, i.e. in all chromatography steps, conditions are first adjusted to allow binding of the bioconjugate to a chromatography medium and subsequently adjusted to allow elution of the bioconjugate from said chromatography medium (bind-elute mode).

It is to be understood that steps i-v are performed in the order as indicated. When following this purification scheme, the eluate after each chromatography step is enriched for the O-EPA bioconjugate compared to the eluate of the previous step, i.e. the purity of O-EPA bioconjugate is increased after each chromatography step compared to the previous one.

The individual production steps outlined above are known per se when looked at individually. However, it was found that the specific combination of steps i-v described above is particularly suitable for the production of a variety of different O-EPA bioconjugates and is amenable to large scale production.

The inventive process yields an O-EPA bioconjugate typically with a purity ≥90%, e.g. 90%, 91%, 92%, 93%, 94%, 95%, 96%, 97%, 98%, 99%, 99.5%, 99.7%, 99.9% or 100% as measured by SE-HPLC or with a purity ≥95%, e.g. 95%, 96%, 97%, 98%, 99%, 99.5%, 99.7%, 99.9% or 100% as measured by RP-HPLC. Typically, the purity of the O-EPA bioconjugate is ≥95%, preferably ≥98%, e.g. 98%, 99%, 99.5%, 99.7% 99.9% or 100% as measured by either of SE-HPLC or RP-HPLC. A purity of at least 90% or higher as measured by SE-HPLC, e.g. at least 95% or at least 98%, is advantageous for components of pharmaceutical products that are intended for administration to humans, such as multivalent bioconjugate vaccines comprising multiple O-EPA conjugates.

Moreover, when following the above protocol, the overall yield of O-EP A bioconjugate is acceptable for a large scale process for making pharmaceutical preparations of O-EPA bioconjugates (e.g. at least 5% overall yield from a bioreactor of at least 150 L, preferably at least 10% overall yield, e.g. about 5-35% overall yield [relative to O-EPA bioconjugate present in a filtered periplasmic fraction], with a purity of at least 90% as measured by SE-HPLC). Typically the purity is at least 95%, as measured by either of SE-HPLC or RP-HPLC.

Importantly and surprisingly, the specific combination of steps i-v typically yields O-EPA bioconjugate compositions with a relatively high proportion of multi-glycosylated O-EPA bioconjugates.

Further details on each of steps i-v are provided below:

Step i: In a first step, an FPF of Gram-negative bacterial host cells that express the O-EPA bioconjugate is provided. As described above, the FPF comprises the O-EPA bioconjugate.

Providing a filtered periplasmic host cell fraction comprising the O-EPA bioconjugate is known per se (see e.g. WO 2009/104074, WO 2015/124769 or WO 2020/191082). To obtain a periplasmic fraction by osmotic shock treatment, cells are first incubated in a buffer with relatively high osmolarity (hypertonic) and then incubated in a buffer with relatively low osmolarity (hypotonic). Such osmotic shock treatment leads to at least partial removal of the cell wall and generation of spheroblasts (i.e. cells, particularly Gram-negative cells, from which the cell wall has been at least partly removed). Thus, the majority of host cell proteins remains in the spheroblasts whereas periplasmic proteins are liberated into the suspension medium.

Typically, E. coli host cells comprising genetic information encoding PglB, EPA and the enzymes for the biosynthesis of the corresponding O-antigen polysaccharide are cultured in a bioreactor, e.g. having a volume of about 100 L-20000 L, e.g. 150 L-5000 L such as 200 L or 800 L or 2000 L, wherein the cells produce the O-EPA bioconjugate. Typically, cells are harvested when they are in the stationary phase, upon cooling the culture to below 20° C., via centrifugation, e.g. via continuous centrifugation e.g. using a disc stack centrifuge. Cells are resuspended in a suitable liquid, e.g. 0.9% NaCl solution or Tris-buffered saline (TBS), and subjected to osmotic shock, preferably using solutions at about 2-15° C., preferably about 6-10° C. Osmotic shock can for instance be performed by addition of sucrose solution (e.g. 60% sucrose, pH 8, 480 mM Tris-HCl, 24 mM EDTA) to the cells to a target concentration of sucrose of about 20-30%, e.g. 25%, incubation of the mixture at about 2-15° C., preferably about 6-10° C. for about 15 minutes to 4 hours, e.g. about 1 hour, while mixing. After the incubation with sucrose the cell/sucrose solution is mixed with a solution of low osmotic value, e.g. by mixing with about 4× the volume of 10 mM Tris-HCL (pH 8.0), at 6-10° C. Mixing can for instance be performed through a static mixer. The O-EPA product is released from the periplasmic space into the supernatant (referred to as periplasmic fraction, PF), and the PF is collected. The material may then be clarified, e.g. by re-moving cell debris in the PF by separation in a disc stack centrifuge, whereby the supernatant (referred to as centrifuged periplasmic fraction, CPF) is collected. The CPF is preferably further filtered to remove remaining cell debris, e.g. by filtration through a depth and bioburden reduction filter (e.g. a membrane with a pore size of about 0.2 μm), and the resulting material is collected as filtered periplasmic fraction (FPF). This FPF can subsequently be used in the chromatographic purification process [starting at step ii] of the invention as described herein.

Thus, in an embodiment, step i further comprises (i-1) incubation of a Gram-negative bacterial host cell in a bioreactor with a volume of between 100 L and 20000 L, e.g. from 150 L to 5000 L, such as 200 L or 800 L or 2000 L, at a temperature between 34° C. and 36° C., e.g. 35° C., to grow until stationary phase prior to harvest; followed by (i-2) harvesting the Gram-negative bacterial host cells, wherein harvesting comprises a continuous flow centrifugation step to obtain harvested Gram-negative bacterial host cells comprising O-EPA bioconjugate in the periplasm.

Thus, in another embodiment, step i further comprises (i-3), osmotic shock treatment of the host cells to obtain a periplasmic fraction of Gram-negative bacterial host cells comprising O-EPA bioconjugate. It is clear to the skilled person that step (i-3) follows step (i-2). In certain embodiments, osmotic shock treatment in step (i-3) comprises addition of sucrose solution preferably further comprising EDTA to the cells to a target concentration of about 25% sucrose, incubation of the mixture at about 6-10° C. for about 15 minutes to 2 hours while mixing, and subsequently adding a solution having low osmolality (e.g. 10 mM Tris-HCl pH 8) at about 6-10° C. to the cell/sucrose solution to bring down the osmotic value by a factor of at least four (as compared to the composition comprising 25% sucrose), whereby the O-EPA bioconjugate is released from the periplasm into the supernatant, and collecting the supernatant (periplasmic fraction), and subsequently filtering the periplasmic fraction to obtain a filtered periplasmic fraction.

In a further embodiment, step i additionally comprises step (i-4) filtration to obtain a filtered periplasmic fraction (FPF) comprising the O-EPA bioconjugate. As outlined above, the filtration step serves to remove remaining cell debris, e.g. by filtration through a depth and bioburden reduction filter (e.g. a membrane with a pore size of about 0.2 μm).

In one embodiment, step (i) comprises
(i-1) incubation of the host cells in a bioreactor with a volume of between 100 L and 20000 L, e.g. from 150 L to 5000 L, at a temperature between 34° C. and 36° C. to grow until stationary phase prior to harvest;
(i-2) harvesting the host cells by a continuous flow centrifugation step to obtain harvested host cells comprising bioconjugate; and
(i-3) osmotic shock treatment of the host cells, to thereby obtain a periplasmic fraction of the host cells, the periplasmic fraction comprising the bioconjugate; and
(i-4) filtration to obtain a filtered periplasmic fraction (FPF) comprising the O-EPA bioconjugate.

Host cell: In a preferred embodiment, the Gram-negative bacterial host cells from which the O-EPA bioconjugate is obtained comprise genetic information encoding (a) a bacterial O-antigen polysaccharide and (b) a recombinant Pseudomonas aeruginosa ExoProtein A (EPA) that comprises at least one glycosylation site and (c) a metabolic apparatus that carries out N-glycosylation of the EPA with the O-antigen polysaccharide thereby producing the O-EPA conjugate in vivo in the periplasm of the Gram-negative bacterial host cells.

In a preferred embodiment, the Gram-negative bacterial host cell is an Escherichia coli host cell, more preferably an E. coli K-12 host cell such as E. coli K-12 strain W3110. Details of this aspect are described in more detail below, and in the art, e.g. in WO 2009/104074, WO 2020/191082 and WO2020/191088 (included herein by reference in its entirety).

In a preferred embodiment, the waaL gene is deleted from or functionally inactivated in the genome of a host cell of the invention. The terms “waaL” and “waaL gene” refer to the O-antigen ligase gene encoding a membrane bound enzyme with an active site located in the periplasm. The waaL gene encoded enzyme transfers un-decaprenylphosphate (UPP)-bound O antigen to the lipid A core, forming lipopolysac-charide. Deletion or disruption of the endogenous waaL gene (e.g., ΔwaaL strains) disrupts transfer of the O-antigen to lipid A, and can instead enhance transfer of the O-antigen to another available biomolecule, such as a carrier protein expressed in a host cell of the invention.

In one embodiment of a host cell of the invention, the E. coli gtrABS genes, which are responsible for O16 O-antigen glucosylation, are deleted from or functionally inactivated in the host cell's genome. In a preferred embodiment, the E. coli gtrABS genes are deleted from or functionally inactivated in the genome of an E. coli W3110 host cell. While the gtrA and gtrB genes in different serotypes are highly homologous and interchangeable, the gtrS gene encodes a serotype-specific O-antigen glycosyl transferase. In E. coli W3110 GtrS can transfer a glucose (Glc) residue to the Glc NAc sugar in the a-L-Rha-(1→3)-D-GlcNAc motif of the E. coli O16 O-antigen.

O-antigen polysaccharide: In a preferred embodiment, the O-antigen polysaccharide is specific to a Gram-negative bacterium selected from the list consisting of Escherichia and Shigella, preferably E. coli.

In one embodiment, the O-antigen polysaccharide is selected from the group consisting of E. coli O-antigen polysaccharide O1A, O2, O4, O8, O6A, O15, O16, O18A, O25B and O75.

In one embodiment, the O-antigen polysaccharide is selected from the group consisting of E. coli O-antigen polysaccharide O1A, O2, O4, O6A, O15, O16, O18A, O25B and O75.

In one embodiment, the O-antigen polysaccharide is selected from the group consisting of E. coli O-antigen polysaccharide O1A, O2, O6A, O16, O18A, O25B and O75. In one embodiment, the O-antigen polysaccharide is selected from the group consisting of E. coli O-antigen polysaccharide O1A, O2, and O25B.

In one embodiment the O-antigen polysaccharide is E. coli O-antigen polysaccharide O1A.

In one embodiment the O-antigen polysaccharide is E. coli O-antigen polysaccharide O2.

In one embodiment the O-antigen polysaccharide is E. coli O-antigen polysaccharide O25B.

In E. coli, the gene products involved in O-antigen polysaccharide biogenesis are encoded by the rfb locus. The host cell as herein provided thus further preferably comprises a nucleotide sequence of an E. coli rfb locus corresponding to an E. coli serotype selected from the list consisting of O1A, O2, O4, O6A, O8, O15, O16, O18A, O25B, and O75.

As used herein, “O-antigen rfb locus” and “O-antigen rfb gene cluster” refer to a locus in the Gram-negative bacterial genome that comprises a cluster of genes that together encode the enzymatic machinery capable of synthesizing an O-antigen polysaccharide structure. The term rfb locus preferably refers to a genomic locus from the genus Escherichia, particularly E. coli.

In certain embodiments, the O-antigen rfb locus is heterologous to the host cell, e.g. introduced into a precursor cell of the host cell, and preferably integrated into the genome thereof. Preferably an original rfb gene cluster, if such was present in a precursor cell, has been replaced by the O-antigen rfb gene cluster in the host cell, to enable production of a bioconjugate of O-antigen polysaccharides preferably selected from the list consisting of E. coli O1A, O2, O4, O6A, O8, O15, O16, O18A, O25B, and O75-antigen polysaccharides.

Exemplary sequences for rfb gene clusters (rfb loci) that can be used in production strains for bioconjugates with O-antigen polysaccharides of the E. coli serotypes listed above are given below:

In one exemplary and non-limiting embodiment, the rfb locus encoding O-antigen polysaccharide of E. coli serotype O1A has the sequence of SEQ ID NO: 11 of WO 2020/191082.

In one exemplary and non-limiting embodiment, the rfb locus encoding O-antigen polysaccharide of E. coli serotype O2 has the sequence of SEQ ID NO: 12 of WO 2020/191082.

In one exemplary and non-limiting embodiment, the rfb locus encoding O-antigen polysaccharide of E. coli serotype O4 has the sequence of SEQ ID NO: 9 of WO 2020/191082. In one exemplary and non-limiting embodiment, a production strain that comprises the rfb locus encoding O-antigen polysaccharide of E. coli serotype O4, further comprises nucleic acid encoding the E. coli O4-specific GtrS protein, this protein for example having SEQ ID NO: 4 of WO 2020/191082.

In one exemplary and non-limiting embodiment, the rfb locus encoding O-antigen polysaccharide of E. coli serotype O6A has the sequence of SEQ ID NO: 13 of WO 2020/191082.

In one exemplary and non-limiting embodiment, the rfb locus encoding O-antigen polysaccharide of E. coli serotype O8 has the sequence of SEQ ID NO: 14 of WO 2020/191082.

In one exemplary and non-limiting embodiment, the rfb locus encoding O-antigen polysaccharide of E. coli serotype O15 has the sequence of SEQ ID NO: 15 of WO 2020/191082.

In one exemplary and non-limiting embodiment, the rfb locus encoding O-antigen polysaccharide of E. coli serotype O16 has the sequence of SEQ ID NO: 16 of WO 2020/191082.

In one exemplary and non-limiting embodiment, the rfb locus encoding O-antigen polysaccharide of E. coli serotype O18A has the sequence of SEQ ID NO: 5 of WO 2022/208430, wherein preferably the Wzy O-antigen polymerase that is encoded in said rfb locus has the amino acid sequence of SEQ ID NO: 1 of WO 2022/208430 except that said amino acid sequence comprises isoleucine at a position that corresponds to position 199, lysine at a position that corresponds to position 377 and alanine at a position that corresponds to position 395 in said amino acid sequence. In one exemplary and non-limiting embodiment, the rfb locus encoding O-antigen polysaccharide of E. coli serotype O25B has the sequence of SEQ ID NO: 18 of WO 2020/191082.

In one exemplary and non-limiting embodiment, the rfb locus encoding O-antigen polysaccharide of E. coli serotype O75 has the sequence of SEQ ID NO: 19 of WO 2020/191082.

The structures of the repeat unit for E. coli O-antigen polysaccharides O1A, O2, O4, O6A, O8, O15, O16, O18A, O25B, and O75 are shown in Table 1.

Optionally, the chain lengths of the O-antigens can be manipulated by manipulating the native Wzz O-antigen chain length regulator mechanism, e.g. by overexpression or supplementing a Wzz O-antigen chain regulator, e.g. by replacing an E. coli wzzB gene with one of its counterparts from species within Salmonella and Shigella, or from P. aeruginosa, e.g. a Salmonella enterica counterpart, fepE or other wzz homolog (see e.g., US 2018/0099038, WO 2020/039359), whereby for instance the number of repeat units of the O-antigen can be increased, with or without additional overexpression of the Wzy protein. The effects of wzz-like genes have been described in literature for these species. None of this is however needed to obtain bioconjugates with good immunogenicity, and in preferred embodiments bioconjugates according to the invention are prepared without manipulation of the Wzz chain length regulator mechanism.

Carrier protein: The carrier protein is detoxified ExoProtein A of P. aeruginosa (EPA), also referred to as recombinant EPA.

For EPA, various detoxified protein variants have been described in literature and could be used as carrier proteins. In certain embodiments, the EPA carrier proteins used in the bioconjugates of the invention are modified in such a way that the protein is less toxic and/or more susceptible to glycosylation. For example, detoxification can be achieved by mutating and deleting the catalytically essential residues L552V and AE553 (see e.g. Lukac et al., 1988, Infect Immun, 56:3095-3098; Ho et al., 2006, Hum Vaccin, 2:89-98). In a specific embodiment, the carrier proteins used in the generation of the bioconjugates of the invention are modified such that the number of glycosylation sites in the carrier proteins is optimized in a manner that allows for lower concentrations of the protein to be administered, e.g., in an immunogenic composition, in its bioconjugate form. In a particular embodiment, the host cell encodes EPA comprising 1-10, preferably 2-4, more preferably 4 glycosylation sites comprising a glycosylation consensus sequence Asn-X-Ser(Thr), wherein X can be any amino acid except Pro, more preferably a glycosylation consensus sequence Asp(Glu)-X-Asn-Z-Ser(Thr), wherein X and Z are independently selected from any amino acid except Pro. Thus, in a preferred embodiment, the carrier protein is recombinant EPA that comprises 4 N-glycosylation sites comprising a glycosylation consensus sequence Asn-X-Ser(Thr), preferably glycosylation consensus sequence Asp(Glu)-X-Asn-Z-Ser(Thr). In one embodiment, the carrier protein comprises an amino acid sequence with at least 80, 85, 90, 95, 96, 97, 98 or 99% sequence identity to SEQ ID NO: 1, and comprises 1-10, preferably 2-4, preferably 4 glycosylation sites comprising a glycosylation consensus sequence Asn-X-Ser(Thr), more preferably a glycosylation glycosylation consensus sequence Asp(Glu)-X-Asn-Z-Ser(Thr). In a preferred embodiment, the carrier protein is recombinant EPA that comprises the amino acid sequence of SEQ ID NO: 1. In a further preferred embodiment, the host cell comprises a nucleotide sequence encoding the carrier protein comprising SEQ ID NO: 1.

Metabolic apparatus for N-glycosylation of EPA: The Gram-negative bacterial host cell preferably comprises an oligosaccharyl transferase (OST) for transfer of oligosaccharides to N-glycosylation sites on the carrier protein. Preferably therefore, a host cell as herein provided comprises a nucleotide sequence encoding an oligosaccharyl transferase (OST). Oligosaccharyl transferases as used herein are enzymes that transfer lipid-linked oligosaccharides to residues of nascent polypeptide chains that comprise a glycosylation consensus motif, for example to asparagine (Asn, N) residues of nascent polypeptide chains that comprise an N-glycosylation consensus motif, examples of such N-glycosylation consensus motifs being motifs having an amino acid of Asn-X-Ser(Thr) or (Asp(Glu)-X-Asn-Z-Ser(Thr)). Throughout this application it is to be understood that for the N-glycosylation motif Asn-X-Ser(Thr), X can be any amino acid except proline, and for the N-glycosylation motif Asp(Glu)-X-Asn-Z-Ser(Thr), both X and Z can be any amino acid except proline. Preferably such oligosaccharyl transferases transfer the oligosaccharides to asparagine residues of the glycosylation consensus sequence glycosylation consensus sequence Asp(Glu)-X-Asn-Z-Ser(Thr) in a polypeptide chain of a carrier protein as described herein. The nucleic acid that encodes an oligosaccharyl transferase can be native to the host cell, or can be introduced into the host cell using genetic approaches. In preferred embodiments, the oligosaccharyl transferase is heterologous to the host cell. E. coli does not naturally comprise an oligosaccharyl transferase, and hence if E. coli is used as a host cell for production of bioconjugates, a heterologous oligosaccharyl transferase is comprised in such host cell, e.g. upon introduction by genetic engineering. The oligosaccharyl transferase can be from any source known in the art in view of the present disclosure.

In certain preferred embodiments, the oligosaccharyl transferase is an oligosaccharyl transferase from Campylobacter. For example, in one embodiment, the oligosaccharyl transferase is an oligosaccharyl transferase from Campylobacter jejuni (i.e., PglB; see, e.g., Wacker et al., 2002, Science 298:1790-1793; see also, e.g., NCBI Gene ID: 3231775, UniProt Accession No. O86154). In another embodiment, the oligosaccharyl transferase is an oligosaccharyl transferase from Campylobacter lari (see, e.g., NCBI Gene ID: 7410986).

In specific embodiments, the oligosaccharyl transferase is PglB from Campylobacter jejuni, including the natural (wild-type) protein or any variant thereof, such as those described in WO 2016/107818 and WO 2016/107819. PglB can transfer lipid-linked oligosaccharides to asparagine residues in the consensus sequences Asn-X-Ser(Thr) and Asp(Glu)-X-Asn-Z-Ser(Thr). In certain embodiments, the PglB oligosaccharyl transferase is a polypeptide having oligosaccharyl transferase activity as defined herein, which polypeptide comprises an amino acid sequence with at least 80, 85, 90, 95, 96, 97, 98 or 99% sequence identity to SEQ ID NO: 2. In a preferred embodiment, the PglB oligosaccharyl transferase comprises an amino acid sequence that is identical to SEQ ID NO: 2, or that is identical to SEQ ID NO: 2 with specified amino acid changes specific to optimize expression of bioconjugates of specific E. coli O-antigen serotypes (see e.g. WO 2020/191088, and further examples in the present application below). Hence, in a preferred embodiment, the host cell comprises a nucleotide sequence encoding the oligosaccharyl transferase comprising SEQ ID NO: 2, or a variant thereof as described herein. In certain embodiments one or more endogenous glycosylation consensus sequences in a wild-type PglB have been mutated to avoid PglB auto-glycosylation, e.g. SEQ ID NO: 2 comprising the mutation N534Q. Examples of variant PglB suitable for use in the host cell provided herein include the PglB of SEQ ID NO: 2 (e.g. for bioconjugates of EPA with E. coli O-antigens of serotypes O2, O8, O18A or O25B); the PglB of SEQ ID NO: 2 comprising the mutation N311V; the PglB of SEQ ID NO: 2 comprising the mutations N311V, K482R, D384H, and A669V (e.g. for bioconjugates of EPA with E. coli O-antigens of serotypes O1A or O15); the PglB of SEQ ID NO: 2 comprising the mutations Y77H and N311V (e.g. for bioconjugates of EPA with E. coli O-antigens of serotype O4); the PglB of SEQ ID NO: 2 comprising the mutations Y77H, S80R, Q287P, K289R and N311V (e.g. for bioconjugates of EPA with E. coli O-antigens of serotypes O6A or O16); and the PglB of SEQ ID NO: 2 comprising the mutations Y77R and N311V (e.g. for bioconjugates of EPA with E. coli O-antigens of serotype O75).

As will be understood by the skilled person, the polypeptide of the invention comprised in a host cell of the invention, such as the carrier protein, oligosaccharyltransferase, and the enzymes of the rfb gene cluster including a polypeptide having Wzy O-antigen polymerase activity as provided e.g. in WO 2020/191088 or WO 2022/208430, are conveniently provided to the host cell in the form of nucleic acids encoding the polypeptides of the invention. The nucleic acids encoding the polypeptides of the invention preferably are nucleic acid constructs, specifically expression constructs comprising one or more expression cassettes for expression of the polypeptides of the invention, wherein nucleotide sequences encoding the polypeptides of the invention are operably linked to expression control sequences for expression of the polypeptides in the host cell of the invention. Suitable expression control sequences usually at least include a promoter and may include further sequence elements such as inter alia a Shine-Dal-garno sequence and a transcription termination sequence. The promoter may be a con-stitutive promoter, or it may be a promoter of which the activity can be regulated, e.g. repressed or induced upon certain conditions, e.g. temperature changes or presence of certain chemicals or proteins in the cell, all of which are as such well known in the art. Non-limiting examples of suitable promoters are ara, phoA, tac, tet, trc, trp, PBAD, APL, T5, or T7 promoter. The nucleic acid constructs can be extrachromosomal, e.g. a plasmid or other vector, or nucleic acid constructs can be integrated into the genome of the host cell of the invention. Molecular biological methods for the construction and/or synthesis of the nucleic acid constructs for expression of the polypeptides in a host cell of the invention are generally well known in the art. In certain embodiments, the rfb locus is integrated into the genome of the host cell. In certain embodiments, nucleic acid encoding the oligosaccharyltransferase and nucleic acid encoding the EPA carrier protein are present on plasmids in the host cell.

Step ii: In a second step the O-EPA bioconjugate is purified by a first anion exchange chromatography (AEX1; step ii). This step serves to reduce the processing volume and to reduce process related impurities derived from fermentation procedures, i.e. incubation of the host cell and extraction of a periplasmic fraction comprising the O-EPA bioconjugate. The main task of the AEX1 is the removal of a large part of DNA, endotoxin and host cell proteins (HCP). In particular, impurities that can lead to the degra-dation of the O-EPA conjugate, such as proteases, peptidases and glycosidases are removed. Thus, in particular, the AEX1 step is a capture step.

In certain embodiments, the AEX1 is performed with a strong anion exchange resin. In certain embodiments, the resin is a ceramic resin, e.g. having rigid, non-compressible characteristics, and has high dynamic binding capacities at high flow rates. A non-limiting example of a resin that is particularly suitable for the AEX1 step is a Q Ceramic HyperD F resin. In certain embodiments, the AEX1-step is performed in bind-elute mode.

Step ii includes subjecting an optionally adjusted load of the FPF to a first anion exchange chromatography (AEX 1) step to obtain a first AEX eluate (AEX1). This step is known per se. Typically, adjusting a load of the filtered periplasmic fraction prior to AEX1 is not required. Instead, the filtered periplasmic fraction is typically directly subjected to AEX1, unless the conditions, in particular the conductivity, of said filtered periplasmic fraction are not suitable for subjecting to AEX1. Thus, adjusting a load of the FPF prior to AEX1 is optional.

In certain embodiments, step ii further comprises one or more of the following steps (ii-1) to (ii-3), preferably in the order as indicated:

In one embodiment, this includes (ii-1) contacting the optionally adjusted load of the filtered periplasmic fraction with the AEX1 medium, and washing said medium comprising bound O-EPA bioconjugate with a wash buffer. Typically, said wash buffer has a relatively low salt concentration and a relatively low conductivity. An example of a suitable low salt, low conductivity wash buffer is a buffer at pH 8.5, comprising 10 mM Tris and 50 mM NaCl.

In one embodiment, this includes (ii-2) eluting the O-EPA bioconjugate with an elution buffer, typically comprising a relatively high salt concentration and a relatively high conductivity. An example of a suitable high salt, high conductivity buffer is a buffer at pH 8.5, comprising 10 mM or 50 mM Tris and 1 M NaCl. Typically, elution is performed using a step gradient.

It will be clear for each of these steps that the identity of the AEX1 resin, AEX1 column or membrane or cassette format, the exact buffer components, pH, and/or salt concentration or conductivity as well as the gradient may be varied, as is known to the skilled person based upon the current disclosure.

In one embodiment, this includes (ii-3) optionally pooling fractions with an enriched O-EPA bioconjugate content to obtain an AEX1 eluate. Typically, fractions with an enriched O-EPA conjugate content are identified by performing an SDS-PAGE analysis, or any suitable analysis to determine the amount or relative purity of the desired O-EPA conjugate. Performing SDS-PAGE analysis to thereby identify fractions comprisesing the intended product, i.e. O-EPA bioconjugate, is well-known to the skilled person. It will be clear to the skilled person that alternatively to pooling fractions based upon analysis of the content of these fractions, it is also possible to simply collect a larger fraction during elution that is known to contain the product, especially once the process steps have been established and experience has been obtained with the elution profile of the product.

Step iii: In a third step, the O-EPA bioconjugate is further purified by subjecting an optionally adjusted load of the AEX1 eluate to a mixed-mode chromatography (MMC) step on a multimodal resin (MMR) to obtain an MMR eluate, said MMR comprising both, anion exchange functionalities and hydrophobic functionalities. This chromatography step further removes process-related impurities, especially endotoxins and host cell proteins (HCP). It was surprisingly found that an MMR comprising both anion exchange functionalities and hydrophobic functionalities, is particularly suitable for separation of bioconjugates as described herein. The use of this step importantly al-lowed to obtain O-EPA bioconjugate compositions with increased levels of multi-glycosylation as compared to a previously preferred process, as shown herein. In addition, the use of this MMR reduced the overall buffer consumption of the total purification process, which is considered beneficial due to reduced costs and a reduced burden on the environment (less waste). This is particularly relevant for a large scale production process, e.g. incubation of the host cells in a bioreactor with a volume of between 100 L and 20000 L, e.g. from 150 L to 5000 L, such as 200 L or 800 L or 2000 L. Various MMRs comprising anion exchange functionalities and hydrophobic functionalities (i.e. comprising cationic and hydrophobic groups) are commercially available, non-limiting examples including PPA HyperCel, HEA HyperCel, MEP HyperCel, and Capto adhere. In certain embodiments, the MMR is Capto adhere.

In a preferred embodiment, said MMR comprises a ligand of formula (I),

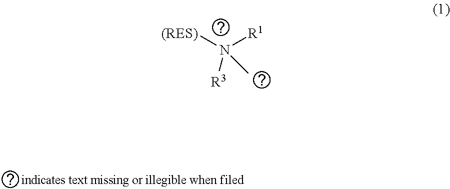

wherein
RES represents the resin of the stationary phase;
R¹ represents C₁-C₄ alkyl, preferably methyl;
R² represents C₁-C₄ alkyl substituted with phenyl, tolyl or xylyl, preferably benzyl;
R³ represents C₁-C₄ alkyl substituted with a hydroxyl or thiol group, preferably 2-hy-droxyethyl.

An example of a suitable ligand of formula (I) is N-benzyl-N-methylethanolamine. In a preferred embodiment, the MMR is a compressible resin. A compressible resin as described herein has the advantage of a relatively higher resistance to abrasion during slurry preparation (mixing of resin with eluent or wash buffer), as compared to a rigid resin. The higher resistance to abrasion leads to a higher re-usability of the resin. Thus, a compressible MMR as described herein is economically favorable.

Suitable compressible MMRs comprising both anion exchange functionalities and hydrophobic functionalities are commercially available, e.g. Capto adhere.

Adjusting a load of the AEX1 eluate prior to MMC is optional. Typically, adjusting a load of the AEX1 eluate prior to the MMC step is not required. Instead, the AEX1 eluate is typically directly subjected to MMC. In alternative embodiments, a load of the AEX1 eluate is adjusted. For example, pH and conductivity are adjusted to a pre-determined value by adding a suitable buffer, e.g. to a pH of 7.2±0.2 using a buffer comprising 50 mM BisTris at pH 6.0 and in a subsequent step to a target conductivity of 7.5-9 mS/cm using a buffer comprising 10 mM BisTris, pH 7.0. In a preferred embodiment, MMC step is performed in bind-elute mode. Step iii is performed after step ii.

In certain embodiments, step iii further comprises one or more of the following steps (iii-1) to (iii-4), preferably in the order as indicated:

In one embodiment, this includes (iii-1) performing a particle reduction filtration 1. This step leads to a removal of viable and non-viable particles.

In one embodiment, this includes (iii-2), contacting the optionally adjusted AEX1 eluate with the MMR and subjecting said MMR comprising the bound O-EPA bioconjugate to several wash steps. Typically, in a first wash step, the MMR is washed with a buffer having a relatively low conductivity. An example of a suitable low conductivity wash buffer is a buffer comprising 50 mM BisTris, 50 mM NaCl, pH 6.0. Typically, in a second wash step, the MMR is washed with a buffer having a higher conductivity and lower pH than the first wash buffer. An example of a suitable second wash buffer is a buffer comprising 50 mM acetate, 250 mM NaCl, pH 4.7. Typically, in a third wash step, the MMR is washed again with a buffer having a relatively low conductivity, such as the low conductivity wash buffer used for the first wash step, e.g. a buffer comprising 50 mM BisTris, 50 mM NaCl, pH 6.0.

In one embodiment, this includes (iii-3), eluting the O-EPA bioconjugate with a buffer having a relatively high conductivity. An example of a suitable high conductivity elution buffer is an elution buffer comprising 50 mM BisTris, 2 M NaCl, pH 6.0.

In one embodiment, this includes (iii-4), pooling of fractions with an enriched O-EPA bioconjugate content to obtain an MMR eluate. In a preferred embodiment, a fixed volume of fractions is pooled to obtain an MMR eluate with an enriched O-EPA bioconjugate content rather than pooling based on SDS-PAGE analysis of individual fractions. Of course, rather than pooling of fractions it is alternatively possible to simply collect one fraction during elution that is known to contain the product, especially once the process steps have been established and experience has been obtained with the elution profile of the product.

It will be clear for each of these steps that the identity of the MMR, column format, the exact buffer components, pH, and/or salt concentration or conductivity as well as the gradients may be varied, as is known to the skilled person based upon the current disclosure.

Step iv: In a fourth step, the O-EPA bioconjugate is further purified by subjecting an adjusted load of the MMR eluate to a hydrophobic interaction chromatography (HIC) step to obtain a HIC eluate comprising the bioconjugate. The HIC step serves to further remove HCPs, especially non-glycosylated EPA and a variety of mid-sized HCPs (ca. 40-60 kDa).

In certain embodiments, step iv, “subjecting an adjusted load of the MMR eluate obtained in step (iii) to a hydrophobic interaction chromatography (HIC) step to obtain a HIC eluate” further comprises one or more of the following steps (iv-1) to (iv-5), preferably in the order as indicated.

In one embodiment, this includes (iv-1), adjusting the conductivity of a load of the MMR eluate. Conductivity is adjusted using a loading buffer that is suitable for binding to a HIC medium, the loading buffer typically having a relatively high conductivity. An example of a suitable loading buffer is a buffer comprising 2 M K-phosphate at pH 7.0. Typically, adjusting a load of the MMR eluate is performed by adding a weight of loading buffer that is three times the weight of the MMR eluate. A non-limiting example of a hydrophobic interaction medium that can be used for HIC of the invention is a Sartobind Phenyl adsorber, such as a Sartobind Phenyl Jumbo 5 L capsule.

In one embodiment, this includes (iv-2), performing a particle reduction filtration 2 for the removal of viable and non-viable particles.

In one embodiment, this includes (iv-3), contacting the adjusted MMR eluate with the HIC medium and washing said medium comprising the bound O-EPA bioconjugate with a suitable buffer, typically a buffer having a relatively high conductivity, e.g. a buffer comprising 2 M K-phosphate, pH 7.0, and optionally subsequently performing a second wash step with a buffer having a reduced conductivity (for example a mixture comprising 70% of a buffer comprising 2 M K-phosphate, pH 7.0, and 30% WFI water).

In one embodiment, this includes (iv-4), eluting the O-EPA bioconjugate using a suitable elution buffer, typically an elution buffer having a relatively low conductivity.

Preferably, elution is performed with a step gradient using a suitable elution buffer. An example of a suitable buffer to elute the O-EPA bioconjugate is a mixture containing 30% of a buffer comprising 2 M K-phosphate, pH 7.0, and 70% WFI water.

It will be clear for each of these steps that the identity of the HIC resin, column or membrane or cassette format, the exact buffer components, pH, and/or salt concentration or conductivity as well as the gradient may be varied, as is known to the skilled person based upon the current disclosure.

In one embodiment, this includes (iv-5), pooling of fractions with an enriched O-EPA conjugate content to obtain a HIC eluate. It will be clear to the skilled person that alternatively to pooling fractions, it is also possible to simply collect a larger fraction during elution that is known to contain the product, especially once the process steps have been established and experience has been obtained with the elution profile of the product.

Step v: In a fifth step, the O-EPA bioconjugate is further purified by “subjecting an adjusted load of the HIC eluate obtained in step (iv) to a second anion exchange chromatography (AEX2) step to obtain a second AEX eluate as product”. The AEX2 step serves to further remove process related protein impurities, including non-glycosylated EPA, short-chain glycosylated EPA (SEPA; i.e. EPA carrier protein having polysac-charide chains with only about 1-3 repeat units), and, in particular, the E. coli transaldolase B (~37 kDa). Thus, in one aspect the invention provides the use of a polishing step by anion exchange chromatography to reduce the amount of transaldolase B in a preparation comprising an O-EPA bioconjugate. In certain embodiments, the O-EPA bioconjugate has undergone previous purification steps including anion exchange, MMR, and hydrophobic interaction chromatography steps in that order.

AEX medium that is suitable for this AEX2 step is commercially available, and a non-limiting example is a Source Q resin.

In certain embodiments, step (v) further comprises one or more of the following steps (v-1) to (v-4), preferably in the order as indicated.

In one embodiment, this includes (v-1), performing a tangential flow filtration 1 (TFF1) of a load of the HIC eluate to lower the conductivity and lower the pH. The TFF1 step is a diafiltration step. In this step, new buffer is added to the feed at the same rate as the permeate flow rate, i.e. the rate at which the feed passes through the membrane. Thereby the HIC eluate is obtained in a suitable buffer and the volume of the HIC eluate remains constant (buffer exchange). A typically suitable diafiltration volume is for example 5-6 times the volume of the HIC eluate, but this may be adjusted if needed according to routine testing by the skilled person, e.g. of conductivity and pH of the retentate. A suitable buffer for such buffer exchange is for example a buffer at pH 6.0 comprising 10 mM BisTris and 50 mM NaCl.

In one embodiment, this includes (v-2), performing a particle reduction filtration 3 for removal of viable and non-viable particles.

In one embodiment, this includes (v-3), contacting the adjusted load of the filtered HIC eluate with the AEX2 medium and eluting the O-EPA bioconjugate with an elution buffer. In certain embodiments, the elution is performed in particular by a step gradient followed by a linear gradient of increasing salt concentration. Typically, the elution buffer has a relatively high salt concentration and high conductivity. One non-limiting example of a suitable process for eluting the O-EPA bioconjugate is by applying 21% of a buffer containing 10 mM BisTris, 200 mM NaCl, pH 6.0 (buffer V) in buffer U (buffer U containing 10 mM BisTris, 50 mM NaCl, pH 6.0) for 7.5 CV (step gradient), followed by a linear gradient of 21-56% buffer V in buffer U over 7-8, e.g. 7.5 CV.

In one embodiment, this includes (v-4), pooling of fractions with an enriched O-EPA bioconjugate content to obtain an AEX2 eluate as product.

In one embodiment, during step (v), the O-EPA bioconjugate is bound to an AEX2 matrix and eluted by a step gradient followed by a linear gradient of increasing salt concentration as described above, to obtain O-EPA bioconjugate with a purity of at least 90%, preferably at least 95%, more preferably at least 98%, or at least 99%.

It will be clear for each of these steps that the identity of the AEX2 resin, AEX2 column or membrane or cassette format, the exact buffer components, pH, and/or salt concentration or conductivity, the gradients, and a fraction pooling strategy may be varied, as is known to the skilled person based upon the current disclosure.

As outlined above:

In certain embodiments, AEX1 is performed in bind-elute mode.

In certain embodiments, MMC is performed in bind-elute mode.

In certain embodiments, HIC is performed in bind-elute mode.

In certain embodiments, AEX2 is performed in bind-elute mode.

In certain embodiments, AEX1, MMC, HIC and AEX2 are performed in bind-elute mode.

When following the above protocol, in each of the second, third, fourth and fifth step, i.e. in each of the chromatography steps (steps ii-v), the relative amount of O-EPA conjugate versus total protein is higher in the collected eluate than in the load.

In certain embodiments, the production process further comprises an additional step (vi) wherein a load of the AEX2 eluate is adjusted to a pharmaceutically acceptable buffer and concentration thereby obtaining the purified O-EPA conjugate as a pharmaceutical drug substance.

In certain embodiments, step (vi) further comprises one or more of the following steps (vi-1) to (vi-3), preferably in the order as indicated:

In one embodiment, this includes (vi-1), performing a tangential flow filtration 2 (TFF2) of the AEX2 eluate to change to the pharmaceutically acceptable buffer and concentration of the O-EPA bioconjugate. In one embodiment, TFF2 is performed with an excipient buffer comprising 6.19 mM KH₂PO₄, 3.81 mM Na₂HPO₄, 5% (w/w) sorbitol, 10 mM methionine, at pH 7.0. In one embodiment, after TFF2, polysorbate-80 is added to a concentration of 0.02% (w/w) to obtain the purified O-EPA bioconjugate in a pharmaceutically acceptable buffer. Thus, in one embodiment, the pharmaceutically acceptable buffer comprises 6.19 mM KH₂PO₄, 3.81 mM Na₂HPO₄, 5% (w/w) sorbitol, 10 mM methionine, 0.02% (w/w) polysorbate-80 at pH 7.0. A typically suitable diafiltration volume for TFF2 is for example 5-6 times the volume of the AEX2 eluate, but this may be adjusted if needed according to routine testing by the skilled person, e.g. of conductivity and pH of the retentate.

Optionally the pH of a load of the AEX2 eluate is adjusted prior TFF2. A suitable pH for TFF2 is e.g. 6.5±0.2 and one example of a suitable buffer is 100 mM Na₂HPO₄ buffer (Buffer W). Typically and preferably, pH of a load of the AEX2 eluate is not adjusted prior to TFF2 (in which case the pH typically is about 6.0±0.2).

In one embodiment, this includes (vi-2), performing a bioburden filtration of the purified O-EPA bioconjugate to obtain the purified O-EPA bioconjugate drug substance. Such bioburden filtration can for example be performed using a filter with a PES membrane with 0.45+0.2 μm cut-off, for example a Sartopore 2 Capsule Size 9 filter.

In one embodiment, this includes (vi-3), portioning and freezing of the purified O-EPA bioconjugate drug substance to thereby obtain a drug substance bulk.

In certain embodiments, the production process further comprises an additional step (vii), wherein several purified O-EPA bioconjugate drug substances are combined thereby obtaining a multivalent drug product.

In certain embodiments, the multivalent drug product comprises at least four, five, six, seven, eight, nine, or ten, purified O-EPA bioconjugates comprising O-antigen polysaccharides selected from the list consisting of E. coli O-serotypes O1A, O2, O4, O6A, O8, O15, O16, O18A, O25B, and O75. In certain embodiments, the multivalent drug product comprises purified O-EPA bioconjugates comprising O-antigen polysaccharides selected from the list consisting of E. coli O-serotypes O1A, O2 and O25B.

In certain embodiments, the multivalent drug product comprises at least purified O-EPA bioconjugates comprising O-antigen polysaccharides of E. coli O-serotype O1A. In certain embodiments, the multivalent drug product comprises at least purified O-EPA bioconjugates comprising O-antigen polysaccharides of E. coli O-serotype O2. In certain embodiments, the multivalent drug product comprises at least purified O-EPA bioconjugates comprising O-antigen polysaccharides of E. coli O-serotype O25B. In certain embodiments, the multivalent drug product comprises at least purified O-EPA bioconjugates comprising O-antigen polysaccharides of E. coli O-serotypes O1A, O2, O6A and O25B.

In certain embodiments, the multivalent drug product comprises at least four, preferably at least eight, more preferably nine purified O-EPA bioconjugates comprising O-antigen polysaccharides selected from the list consisting of E. coli O-serotypes O1A, O2, O4, O6A, O8, O15, O16, O18A, O25B, and O75, preferably E. coli O-serotypes O1A, O2, O4, O6A, O15, O16, O18A, O25B, and O75. In further embodiments, conjugates of different E. coli serotypes (i.e. O-antigen polysaccharides covalently coupled to carrier protein) may be added, e.g. to obtain a multivalent drug product comprising 10-20 conjugates, e.g. O-EPA conjugates. Such conjugates of different E. coli serotypes may also be bioconjugates, and may also have been purified according to processes described herein. In specific embodiments, a nine-valent drug product that comprises not more than nine purified O-EPA bioconjugates according to the invention is provided and the O-antigen polysaccharides in this nine-valent drug product consist of E. coli O-serotypes O1A, O2, O4, O6A, O15, O16, O18A, O25B, and O75. In specific embodiments, a ten-valent drug product that comprises not more than ten purified O-EPA bioconjugates according to the invention is provided and the O-antigen polysaccharides in this ten-valent drug product consist of E. coli O-serotypes O1A, O2, O4, O6A, O8, O15, O16, O18A, O25B, and O75.

In optional embodiments, additional filtration steps such as tangential flow filtration, ultrafiltration/diafiltration (a variant of tangential flow filtration), dead-end filtration, and/or sterile filtration, as well as optional concentration and/or dilution and/or buffer change steps for the O-EPA bioconjugate may be added at certain points in the process, but preferably no additional chromatography steps are included, and the process thus preferably does not include more than 4 chromatography steps. Preferably a process according to the invention does neither include a size-exclusion chromatography step nor a hydroxyapatite chromatography step for purification of the bioconjugate. Of course further purification steps such as chromatography steps could be added to the methods of the invention to get to even higher purity, but this will inevitably lead to lower overall yields and increased process complexity and material consumption, and is thus not desirable because it will likely make the process economically unfeasible for the large scale production of a biopharmaceutical product, while the process of the invention already leads to the desired purity level for a pharmaceutical product suitable for administration to humans, at a yield that renders the process of the invention economically and practically feasible.

Further details on the process steps and terms used are provided below:

Anion exchange chromatography (AEX): In general, in ion exchange chromatography, binding is based on electrostatic charges. In case of AEX, the resin has positively charged functional groups, thus sample components with negatively charged functional groups will bind to it. AEX can be performed using weak anion exchangers or strong anion exchangers, as known to the skilled person. In certain embodiments of the invention, strong anion exchange resins are used for the AEX steps (AEX1 and AEX2). Non-limiting examples of functional groups that are suitable for AEX resins are quaternary ammonium groups. Such resins are commercially available and include e.g. a Q Ceramic HyperD F resin and a Source 15Q resin. Based upon the present disclosure and common knowledge, the skilled person will know how to vary different available AEX resins for use in a process of the invention. As one non-limiting example AEX1 can suitably be performed with a Q Ceramic HyperD F resin and AEX2 can suitably be performed with a Source 15Q resin. With increasing salt concentration, salt ions in the elution buffer compete for binding with the resin-bound material, and the bound material is displaced and eluted. Alternatively, when pH is changed, bound proteins are ti-trated and eventually become non-charged or have the same charge as the functional groups of the resin, leading to repulsion and elution of the bound protein.

Mixed-mode chromatography: The term mixed-mode chromatography (MMC) is known in the field and used herein synonymously to multimodal chromatography. The abbreviation MMC is not to be confused with the commercially available product “Capto MMC multimodal chromatography resin” (Cytiva). The term relates to chromatographic methods that utilize more than one form of interaction between the stationary phase and the target protein, e.g. the bioconjugate, in order to achieve separation from impurities. Hence, in MMC, binding to a multimodal resin (MMR) and separation of proteins is dependent on at least two different types of interaction. In some cases, binding is determined by a combination of electrostatic and hydrophobic interactions, e.g. in case of MMRs comprising both anion exchange functionalities and hydrophobic functionalities. A non-limiting example of such a resin is an MMR comprising ligands of formula I, e.g. N-benzyl-N-methylethanolamine. Such resins may in particular be compressible resins and are commercially available, e.g. Capto Adhere multimodal resin. In addition, hydrogen bonding may contribute to binding.

However, this type of MMC is distinctly different from hydroxyapatite chromatography (HA), e.g. ceramic HA (cHA), which is sometimes also viewed as a particular form of mixed-mode chromatography. In HA (or cHA) separation is in particular achieved by a combination of ionic and metal-affinity interactions (e.g. a CHT Ceramic Hydroxyapatite XT resin). Typically, protein(s) bind to HA via HA-phosphoryl (cat-ion-exchange) or HA-calcium interactions (metal affinity). Small basic proteins usually bind to HA by phosphoryl-cation exchange and acidic proteins typically interact predominantly by calcium affinity. Large proteins often bind to HA using both mecha-nisms. However, HA (or cHA), albeit being a suitable stationary phase for a wide range of applications, including purification of bioconjugates, does not lead to the superior results for the current application of bioconjugate purification obtained with the MMR according to the invention. For example, use of HA brings higher costs, and moreover the MMR of the present invention importantly and unexpectedly leads to improved multi-glycosylation yields in the bioconjugate compositions prepared according to the processes according to the present invention as compared to similar processes wherein cHA is used instead of MMR [cf. WO 2022/214620].

Hydrophobic interaction chromatography (HIC): In HIC target protein(s), e.g. O-EPA bioconjugate(s), are separated from impurities based on their hydrophobicity. Tar-get protein(s) containing hydrophobic and hydrophilic regions are typically applied to a HIC column in a high-salt buffer. The salt in the buffer reduces the solvation of target protein(s). As solvation decreases, hydrophobic regions that become exposed are ad-sorbed by the HIC resin. The more hydrophobic the molecule, the less salt is needed to promote binding. Usually a decreasing salt gradient is used to elute samples from the column in order of increasing hydrophobicity. Sample elution may also be assisted by the addition of further components, such as detergents, to the elution buffer. A non-limiting example of a HIC capsule that is suitable for performing the above HIC step is a Sartobind Phenyl Jumbo 5L capsule. The skilled person is able to vary the HIC resin and use it according to the invention, based upon the present disclosure and the various commercially available HIC resins.

Bioconjugate: The term is discussed above. Specifically, a bioconjugate is a glycoconjugate prepared in a host cell, wherein the host cell machinery produces the glycan and the carrier protein and links the glycan to the carrier protein, e.g., via N-links of asparagine or arginine. A particularly preferred host cell for producing bioconjugates is E. coli, preferably comprising nucleic acid encoding: (i) the carrier protein, (ii) an oligosaccharyltransferase such as C. jejuni PglB that is capable of covalently linking O-antigen polysaccharides to an asparagine (Asn) residue in a glycosylation consensus sequence (Asn-X-Ser(Thr), wherein X can be any amino acid except Pro) in a carrier protein via N-linked glycosylation, and (iii) an rfb gene cluster encoding the enzymes responsible for generating the O-antigen polysaccharide of a desired serotype. By cre-ating host cells with a different rfb locus, different bioconjugates can be prepared, e.g. comprising O-antigen polysaccharides from different E. coli or Shigella serotypes. Culturing such host cells will produce the bioconjugates comprising the carrier protein to which the O-antigen polysaccharide encoded by the rfb locus is covalently attached, within the periplasm of the host cell. A more detailed description for production of bioconjugates in such host cells can for instance be found in WO 2009/104074, WO 2015/124769, WO 2017/035181, or WO 2020/191082. Optimized variants of the PglB oligosaccharyltransferase for production of bioconjugates of specific E. coli O-antigens has been described in WO 2020/191088. The present invention deals with novel and improved methods of purification of the produced bioconjugates from such host cells as well as bioconjugate compositions obtainable by or obtained from such host cells. The host cell for production of bioconjugates is typically a bacterial cell, preferably a gram-negative bacterial cell, and in preferred embodiments the host cell is E. coli. The O-EPA bioconjugate is thus to be purified from E. coli host cell proteins. One example of a host cell protein is transaldolase B, in particular E. coli transaldolase B, and the processes described in the instant invention are capable of obtaining O-EPA preparations with very low amounts of host cell proteins, including very low amounts of transaldolase B, which surprisingly appeared one of the most abundant residual host cell proteins that was hard to remove from O-EPA bioconjugates. The host cells typically are engineered to express the bioconjugates in the periplasm, and hence a good starting point to purify the O-EPA bioconjugates is from the periplasmic fraction of the host cells, e.g. from gram-negative bacterial host cells, such as E. coli host cells. Particularly useful bioconjugates include carrier proteins to which one or more polysaccharides are attached. Such bioconjugates are for instance used as the active components of certain vaccines, which aim at inducing functional immune responses against the polysaccharides of the bioconjugates. In embodiments of the invention, said bioconjugate comprises one carrier protein and one or more polysaccharides covalently bound to said carrier protein, preferably 1 to 4 polysaccharides covalently bound to said carrier protein.

In embodiments of the invention, the bioconjugate is a conjugation product containing an E. coli O-antigen polysaccharide covalently bound to a carrier protein. In embodiments of the invention, the bioconjugate is a conjugation product containing a Shigella O-antigen polysaccharide covalently bound to a carrier protein. The term O-antigen is known in the field and used in its normal context, it is not to be confused with O-linked. In typical embodiments, the O-antigen polysaccharide is N-linked to the carrier protein. The term O-antigen polysaccharide generally refers to a repetitive glycan polymer contained within an LPS of a bacteria, such as E. coli. The O-antigen of E. coli is a polymer of immunogenic repeating oligosaccharides (typically 1-40 repeating units, e.g. 5-30 repeating units) and typically used for serotyping and glycoconjugate vaccine production.

Carrier Protein: The term is discussed above. A particularly suitable carrier protein in the context of the invention is detoxified Exotoxin A of Pseudomonas aeruginosa (EPA) (the terms Exotoxin A and ExoProtein A of P. aeruginosa, or EPA, are used interchangeably). In particular embodiments, a carrier protein is a detoxified Exotoxin A of P. aeruginosa. For EPA, various detoxified protein variants have been described in literature and could be used as carrier proteins. For example, detoxification can be achieved by mutating and deleting the catalytically essential residues L552V and ΔE553.

Preferably, the EPA carrier protein comprises 1 to 20, preferably 1 to 10, preferably 2 to 4, glycosylation sites.

In certain embodiments, the EPA comprises four glycosylation sites having the amino acid sequence Asn-X-Ser(Thr), preferably Asp(Glu)-X-Asn-Z-Ser(Thr). See for example WO 2015/124769, WO 2017/035181, or WO 2020/191082 for a description of examples of bioconjugation of E. coli O-antigen polysaccharides to EPA carrier protein, or for example WO 2009/104074 for a description of examples of bioconjugation of Shigella O-antigen polysaccharides to EPA carrier protein. In one non-limiting preferred embodiment, the carrier protein of a bioconjugate according to the invention comprises SEQ ID NO: 1. In certain embodiments, during expression in the host cell the carrier protein comprises a signal sequence that targets the carrier protein to the periplasmic space. Various signal sequences can be used. In one non-limiting embodiment, the signal sequence comprises SEQ ID NO: 3. A signal sequence may be cleaved off after translocation of the protein to the periplasm and may thus no longer be present in the final carrier protein of a bioconjugate.

Polysaccharide: The term is discussed above. Suitable polysaccharides comprise 1 to 100, such as 1-50, 1-40, 1-30, 1-20, and 1-10, 3-50, 3-40, e.g. at least 5, such as 5-40, e.g. 7-30, e.g. 7 to 25, e.g. 5 to 20, e.g. 10-20, repeating units n. Such repeating units contain (i.e. comprise or consist of (i) non-modified mono-saccharides and/or (ii) modified monosaccharides. The term “modified mono-saccharides” in non-limiting embodiments includes N-acetylation, O-acetylation, amidation and/or amination of mono-saccharides. Such modified mono-saccharides may comprise zero, one, or more modifications, for example zero, one, two or three of the above modifications, at the same monosaccharide.

In particular embodiments, modified monosaccharides are O-acetylated and/or N-acetylated monosaccharides, specifically monosaccharides comprising one O-acetylation or N-acetylation.

In embodiments of the invention, suitable repeating units comprise mono-saccharides selected from the group consisting of Mannose, Rhamnose, Glucose, Fucose, Galactose, modified Mannose, modified Rhamnose, modified Glucose, modified Fucose, and modified Galactose.

In embodiments of the invention, the O-polysaccharide is specific to a Gram-negative bacterium selected from the list of Escherichia and Shigella, preferably E. coli. Non-limiting and exemplary structures of E. coli O-antigen polysaccharides are shown below in Table 1. A single repeating unit for each E. coli O-antigen polysaccharide is shown. In this table, each n is independently an integer of 1 to 100, such as 1-50, 1-40, 1-30, 1-20, and 1-10, 3-50, 3-40, e.g. at least 5, such as 5-40, e.g. 7-30, e.g. 7 to 25, e.g. 5 to 20, e.g. 10-20, but in some instances can be 1-2. In certain preferred embodiments for bioconjugate compositions of E. coli O-antigen polysaccharides purified according to the methods of the invention, n is on average somewhere between 5-30, preferably 10-25, preferably 10-20.

TABLE 1
Examples of structures of E. coli O-antigen polysaccharides
E. coli O-antigen Polysaccharide
Structure of Repeating Unit
O1A antigen polysaccharide (O1A)

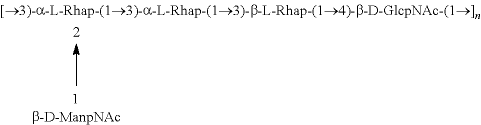

O2 antigen polysaccharide (O2)

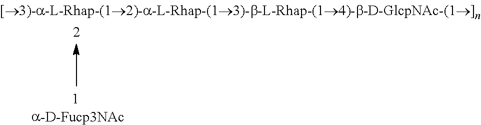

O4 antigen polysaccharide (O4)

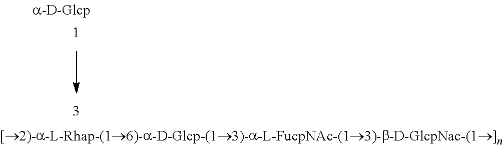

O6A antigen polysaccharide (O6)

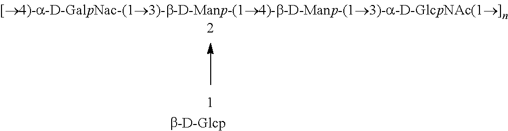

O8 antigen polysaccharide (O8)
α-D-Manp3Me-(1→[3)-β-D-Manp-(1→2)-α-D-Manp-(1→2)-α-D-Manp-(1→]ₙ
O15 antigen polysaccharide (O15)
[→2)-β-D-Galp-(1→3)-α-L-FucpNAc-(1→3)-β-D-GlcpNAc-(1→]ₙ
O16 antigen polysaccharide (O16)

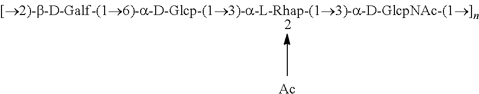

O18A antigen polysaccharide (O18A)

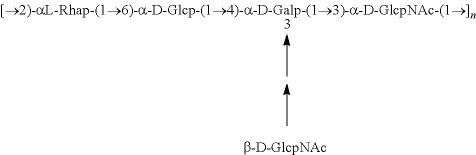

O25B antigen polysaccharide (O25B)

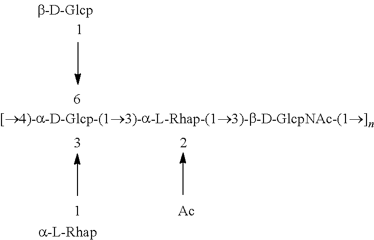

O75 antigen polysaccharide (O75)

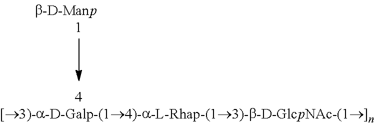

Structures of other E. coli or Shigella or other bacterial O-antigen polysaccharides from various serotypes are known and can be found in the art.

Bioconjugate composition: The term is known in the field and discussed above. In cases where the carrier protein carries multiple glycosylation sites, e.g. EPA carrier protein having SEQ ID NO: 1, production of bioconjugates typically yields a mixture of bioconjugates (herein the bioconjugate composition) rather than a single bioconjugate product.

As used herein, the term “bioconjugate composition” in certain embodiments thus refers to a mixture of bioconjugates, wherein each bioconjugate comprises the same carrier protein and the same O-antigen polysaccharide but individual bioconjugates differ in the number of occupied glycosylation sites (e.g. a “monovalent” composition). In case of EPA carrier protein comprising 4 N-glycosylation sites, e.g. EPA carrier protein comprising four times the glycosylation consensus sequence Asp(Glu)-X-Asn-Z-Ser(Thr), such as EPA carrier protein having SEQ ID NO: 1, a monovalent bioconjugate composition comprises typically several individual bioconjugates each having 1-4 glycans attached to the EPA carrier protein, i.e. mono-, di-, tri- or tetra-glycosylated bioconjugates.

Depending on the individual bioconjugate, particularly O-EPA bioconjugates as defined above, not all individual bioconjugates are necessarily present, e.g. an O-EPA bioconjugate composition may comprise mono-, di- and tri-glycosylated O-EPA bioconjugate but no tetra-glycosylated O-EPA bioconjugate. Nevertheless, O-EPA bioconjugate compositions as defined above may also comprise all four glycosylation forms, i.e. mono-, di-, tri- and tetra-glycosylated O-EPA bioconjugate.

In other embodiments, a bioconjugate composition may be a mixture of several monovalent compositions, i.e. including a mixture of various drug substances that each comprise a carrier protein to which individually O-antigen polysaccharide of a specific bacterial serotype is covalently bound by bioconjugation, wherein in the total composition (a “multivalent” composition) different monovalent compositions are included, so that in the multivalent composition different O-antigen polysaccharides of different bacterial serotypes are included, each individually covalently coupled to a carrier protein. Such a multivalent composition is in certain embodiments also referred to as “drug product”, as it can be used as a vaccine composition to administer to a subject with the aim of inducing an immune response against the bacterial serotypes of which the O-antigen polysaccharides are included in the composition. Each of the individual serotype bioconjugate drug substances present in such multivalent compositions may also be viewed as part of a monovalent composition as indicated above, e.g. in case the EPA carrier protein comprises four N-linked glycosylation sites, for each serotype mono-, di-, tri-, and/or tetra-glycosylated bioconjugates may be present in the multivalent composition.

A bioconjugate composition comprising a carrier protein having SEQ ID NO: 1 (EPA) to which O-antigen polysaccharide of a specific E. coli serotype is covalently attached (O-EPA bioconjugate) thus comprises a mixture of bioconjugates, wherein the O-EPA bioconjugates may be either mono-glycosylated with 1 O-antigen polysaccharide attached to EPA or multi-glycosylated with 2, 3 or 4 O-antigen polysaccharides attached to EPA, so that the bioconjugate composition is a mixture of mono-glycosylated and multi-glycosylated forms of the O-EPA bioconjugate.

Multi-glycosylated bioconjugate forms: As used herein, the term “multi-glycosylated form of a bioconjugate” relates to a bioconjugate comprising a carrier protein to which more than one O-antigen polysaccharide of the same serotype is covalently attached, i.e. more than one N-glycosylation site is occupied. As described above, O-EPA bioconjugates comprising EPA carrier protein with 4 N-glycosylation sites, e.g. EPA carrier protein having SEQ ID NO: 1, may be mono-, di-, tri- or tetra-glycosylated. In this case, the term “multi-glycosylated forms” thus relates to the sum of di-, tri- and tetra-glycosylated forms of the bioconjugate.

Comparison to previous process for production of O-EPA bioconjugates: As described above, WO 2022/214620 describes a process for O-EPA bioconjugate production that is amenable to the large-scale production of bioconjugates. However, the previously described process for production of O-EPA bioconjugates comprising an EPA carrier protein covalently attached to an O-antigen polysaccharide of a specific E. coli serotype yields bioconjugate compositions with a relatively low proportion of multi-glycosylated forms of O-EPA bioconjugates, i.e. EPA carrier protein attached to 2, 3 or 4 O-antigen polysaccharides. Accordingly, pharmaceutical compositions comprising said specific O-EPA bioconjugates necessarily comprise compositions of said specific O-EPA bioconjugates with a relatively low proportion of multi-glycosylated forms of O-EPA bioconjugates.

The degree of glycosylation (DOGY) of O-EPA bioconjugates may be measured by different methods. A well established and particularly suitable method is capillary Gel Electrophoresis (cGE). In cGE, charged molecules are separated in capillaries filled with a porous gel matrix based on their size. CGE is commonly used to separate large biological molecules such as protein or glycoproteins. For example, in case of O-EPA bioconjugates, the presence of covalently bound E. coli O-antigen polysaccharides leads to a change of the mobility of the EPA carrier protein in the capillary. Advantageously, DOGY is measured according to the following protocol:

O-EPA bioconjugates are concentrated to a target concentration of 3 mg/mL and separated in a bare-fused silica capillary (total capillary length of 30.2 cm and an inner diameter of 50 μm) containing a SDS-Gel buffer (pH 8.0, 0.2% SDS, 1% PEG6000), using a voltage of −15 kV. Advantageously, for cGE, absorbance of bioconjugates is measured at 220 nm. The relative amount (peak area percentage) of mono-, di-, tri-, and tetra-glycosylated O-EPA bioconjugate forms is determined by integration of the corresponding peak sections in the electropherogram, to determine the percentage of glycosylated forms. A comparison between the abundance of monoglycosylated and multiglycosylated forms of O-EPA bioconjugates can be established based on the percentage of mono-, di-, tri-, and tetra-glycosylated O-EPA bioconjuagte forms.

A comparison of DOGY as measured by cGE for several exemplary O-EPA bioconjugate compositions produced by either the method according to WO 2022/214620 (example 2) or the inventive method described herein (example 1) showed that the process of the invention leads to a higher proportion of multi-glycosylated forms of O-EPA bioconjugates. This is shown in example 3 (Table 4).

Hence, in a second aspect, the invention relates to O-EPA bioconjugate compositions obtainable or obtained by the inventive production process described herein. In particular, the invention relates to an O-EPA bioconjugate composition comprising a carrier protein having SEQ ID NO: 1 to which O-antigen polysaccharide of E. coli, particularly selected from serotypes O1A, O2, or O25B, is covalently linked, which composition is obtainable or obtained by a method according to the invention. These bioconjugate compositions are particularly suitable for manufacturing the pharmaceutical compositions as described herein.

In certain embodiments, the invention relates to an O-EPA bioconjugate composition obtainable by or obtained by the inventive process described herein, wherein each O-EPA bioconjugate comprises an EPA carrier protein comprising 4 N-glycosylation sites having the amino acid sequence Asn-X-Ser(Thr), preferably having the amino acid sequence Asp(Glu)-X-Asn-Z-Ser(Thr), wherein X and Z are independently selected from any amino acid except Pro, such as the EPA carrier protein comprising the amino acid sequence of SEQ ID NO:1; and to which E. coli O-antigen polysaccharide selected from the group consisting of O1A, O2, O4, O8, O6A, O15, O16, O18A, O25B and O75 is covalently linked. In certain embodiments, the E. coli O-antigen polysaccharide is selected from the group consisting of O1A, O2, O6A, O16, O18A, O25B and O75. In certain embodiments, the E. coli O-antigen polysaccharide is selected from the group consisting of O1A, O2, and O25B.

In certain embodiments, the invention relates to an O-EPA bioconjugate composition obtainable by or obtained by the inventive process described herein, selected from the group consisting of:
(a) a bioconjugate composition of O-EPA bioconjugate of E. coli serotype O1A, wherein at least 50% is multi-glycosylated;
(b) a bioconjugate composition of O-EPA bioconjugate of E. coli serotype O2, wherein at least 44% is multi-glycosylated;
(c) a bioconjugate composition of O-EPA bioconjugate of E. coli serotype O6A, wherein at least 64% is multi-glycosylated;
(d) a bioconjugate composition of O-EPA bioconjugate of E. coli serotype O16, wherein at least 65% is multi-glycosylated;
(e) a bioconjugate composition of O-EPA bioconjugate of E. coli serotype O18A, wherein at least 70% is multi-glycosylated;
(f) a bioconjugate composition of O-EPA bioconjugate of E. coli serotype O25B, wherein at least 50% is multi-glycosylated; and/or
(g) a bioconjugate composition of O-EPA bioconjugate of E. coli serotype O75, wherein at least 50% is multi-glycosylated, wherein each of the percentages for multi-glycosylated and mono-glycosylated O-EPA are determined by capillary gel electrophoresis.

It is understood that the definitions, terms, characteristics, parameters disclosed herein (specifically of the first aspect of the invention) apply likewise to this second aspect.

In a third aspect, the invention relates to specific pharmaceutical compositions comprising one or more bioconjugate compositions as defined herein. This aspect particularly provides pharmaceutical compositions comprising bioconjugate compositions wherein EPA carrier protein is covalently attached to one of O-antigen polysaccharides of E. coli serotypes selected from the group consisting of O1A, O2, O6A, O15, O16, O18A, O25B or O75. These pharmaceutical compositions show particularly beneficial amounts of multi-glycosylated forms of EPA carrier protein. This aspect is explained in further detail below. It is understood that the definitions, terms, characteristics, parameters disclosed herein (specifically in the first aspect of the invention) apply likewise to this third aspect.

In a preferred embodiment, the pharmaceutical composition comprises one or more bioconjugate compositions. Each of these bioconjugate compositions comprises a Pseudomonas aeruginosa ExoProtein A carrier protein (EPA) having SEQ ID NO: 1 to which O-antigen polysaccharide of a specific E. coli serotype is covalently attached (O-EPA bioconjugate). Each of the O-EPA bioconjugates may be either mono-glycosylated with 1 O-antigen polysaccharide attached to EPA or multi-glycosylated with 2, 3 or 4 O-antigen polysaccharides attached to EPA, so that the bioconjugate composition is a mixture of mono-glycosylated and multi-glycosylated forms of the O-EPA bioconjugate. In particular, the pharmaceutical composition comprises one or more of the following:
(a) a bioconjugate composition of O-EPA bioconjugate of E. coli serotype O1A, wherein at least 50% is multi-glycosylated;
(b) a bioconjugate composition of O-EP A bioconjugate of E. coli serotype O2, wherein at least 44% is multi-glycosylated;
(c) a bioconjugate composition of O-EPA bioconjugate of E. coli serotype O6A, wherein at least 64% is multi-glycosylated;
(d) a bioconjugate composition of O-EPA bioconjugate of E. coli serotype O16, wherein at least 65% is multi-glycosylated;
(e) a bioconjugate composition of O-EPA bioconjugate of E. coli serotype O18A, wherein at least 70% is multi-glycosylated;
(f) a bioconjugate composition of O-EPA bioconjugate of E. coli serotype O25B, wherein at least 50% is multi-glycosylated; and/or
(g) a bioconjugate composition of O-EPA bioconjugate of E. coli serotype O75, wherein at least 50% is multi-glycosylated, wherein each of the percentages for multi-glycosylated and mono-glycosylated O-EPA are determined by cGE.

Further details on bioconjugate compositions (a)-(g) are provided below (DOGY measurements by cGE as described above):

Ad composition (a): In an embodiment, the bioconjugate composition of O-EPA bioconjugate of E. coli serotype O1A comprises at least 50% multi-glycosylated O-EPA and not more than 80% multi-glycosylated O-EPA. Thus, in certain embodiments, bioconjugate composition (a) comprises between 50-80% multi-glycosylated O-EPA, e.g. 50%, 55%, 60%, 65%, 67%, 70%, 75% or 80% multi-glycosylated O-EPA. In an embodiment, bioconjugate composition (a) comprises at least 55% multi-glycosylated O-EPA. Furthermore, in certain embodiments, bioconjugate composition (a) comprises at least 20% mono-glycosylated O-EPA and not more than 50% mono-glycosylated O-EPA. Thus, in certain embodiments, bioconjugate composition (a) comprises between 20-50% mono-glycosylated O-EPA, e.g. 20%, 25%, 30%, 35%, 40%, 45% or 50% mono-glycosylated O-EPA.

Ad composition (b): In an embodiment, the bioconjugate composition of O-EPA bioconjugate of E. coli serotype O2 comprises at least 44% multi-glycosylated O-EPA and not more than 90% multi-glycosylated O-EPA. Thus, in certain embodiments, bioconjugate composition (b) comprises between 44-90% multi-glycosylated O-EPA, e.g. 44%, 45%, 50%, 55%, 60%, 65%, 70%, 71%, 75%, 80%, 85% or 90% multi-glycosylated O-EPA. In certain embodiments, bioconjugate composition (b) comprises at least 50% multi-glycosylated O-EPA. Furthermore, in certain embodiments, bioconjugate composition (b) comprises at least 10% mono-glycosylated O-EPA and not more than 56% mono-glycosylated O-EPA. Thus, in certain embodiments, bioconjugate composition (b) comprises between 10-56% mono-glycosylated O-EPA, e.g. 10%, 15%, 20%, 25%, 30%, 35%, 40%, 45%, 50%, 55% or 56% mono-glycosylated O-EPA.

Ad composition (c): In an embodiment, the bioconjugate composition of O-EPA bioconjugate of E. coli serotype O6A comprises at least 64% multi-glycosylated O-EPA and not more than 90% multi-glycosylated O-EPA. Thus, in certain embodiments, bioconjugate composition (c) comprises between 64-90% multi-glycosylated O-EPA, e.g. 64%, 65%, 70%, 72%, 75%, 80%, 85% or 90% multi-glycosylated O-EPA. In certain embodiments, bioconjugate composition (c) comprises at least 70% multi-glycosylated O-EPA. Furthermore, in certain embodiments, bioconjugate composition (c) comprises at least 10% mono-glycosylated O-EPA and not more than 36% mono-glycosylated O-EPA. Thus, in certain embodiments, bioconjugate composition (c) comprises between 10-36% mono-glycosylated O-EPA, e.g. 10%, 15%, 20%, 25%, 30%, 35% or 36% mono-glycosylated O-EPA.

Ad composition (d): In an embodiment, the bioconjugate composition of O-EPA bioconjugate of E. coli serotype O16 comprises at least 65% multi-glycosylated O-EPA and not more than 90% multi-glycosylated O-EPA. Thus, in certain embodiments, bioconjugate composition (d) comprises between 65-90% multi-glycosylated O-EPA, e.g. 65%, 70%, 75%, 80%, 81%, 85% or 90% multi-glycosylated O-EPA. In certain embodiments, bioconjugate composition (d) comprises at least 70% multi-glycosylated O-EPA. Furthermore, in certain embodiments, bioconjugate composition (d) comprises at least 10% mono-glycosylated O-EPA and not more than 35% mono-glycosylated O-EPA. Thus, in certain embodiments, bioconjugate composition (d) comprises between 10-35% mono-glycosylated O-EPA, e.g. 10%, 15%, 20%, 25%, 30% or 35% mono-glycosylated O-EPA.

Ad composition (e): In an embodiment, the bioconjugate composition of O-EPA bioconjugate of E. coli serotype O18A comprises at least 70% multi-glycosylated O-EPA and not more than 95% multi-glycosylated O-EPA. Thus, in certain embodiments, bioconjugate composition (e) comprises between 70-95% multi-glycosylated O-EPA, e.g. 70%, 75%, 76%, 80%, 85%, 90% or 95% multi-glycosylated O-EPA. In certain embodiments, bioconjugate composition (e) comprises at least 80% multi-glycosylated O-EPA. Furthermore, in certain embodiments, bioconjugate composition (e) comprises at least 5% mono-glycosylated O-EPA and not more than 30% mono-glycosylated O-EPA. Thus, in certain embodiments, bioconjugate composition (e) comprises between 5-30% mono-glycosylated O-EPA, e.g. 5%, 10%, 15%, 20%, 25% or 30% mono-glycosylated O-EPA.

Ad composition (f): In an embodiment, the bioconjugate composition of O-EPA bioconjugate of E. coli serotype O25B comprises at least 50% multi-glycosylated O-EPA and not more than 90% multi-glycosylated O-EPA. Thus, in certain embodiments, bioconjugate composition (f) comprises between 50-90% multi-glycosylated O-EPA, e.g. 50%, 55%, 60%, 65%, 70%, 75%, 80%, 85%, 88% or 90% multi-glycosylated O-EPA. In certain embodiments, bioconjugate composition (f) comprises at least 60% multi-glycosylated O-EPA. Furthermore, in certain embodiments, bioconjugate composition (f) comprises at least 10% mono-glycosylated O-EPA and not more than 50% mono-glycosylated O-EPA. Thus, in certain embodiments, bioconjugate composition (f) comprises between 10-50% mono-glycosylated O-EPA, e.g. 10%, 15%, 20%, Ad composition (g): In an embodiment, the bioconjugate composition of O-EPA bioconjugate of E. coli serotype O75 comprises at least 50% multi-glycosylated O-EPA and not more than 90% multi-glycosylated O-EPA. Thus, in certain embodiments, bioconjugate composition (g) comprises between 50-90% multi-glycosylated O-EPA, e.g. 50%, 55%, 60%, 65%, 66%, 70%, 75%, 80%, 85% or 90% multi-glycosylated O-EPA. In certain embodiments, bioconjugate composition (g) comprises at least 60% multi-glycosylated O-EPA. Furthermore, in certain embodiments, bioconjugate composition (h) comprises at least 10% mono-glycosylated O-EPA and not more than 50% mono-glycosylated O-EPA. Thus, in certain embodiments, bioconjugate composition (g) comprises between 10-50% mono-glycosylated O-EPA, e.g. 10%, 15%, 20%, 25%, 30%, 35%, 40%, 45% or 50% mono-glycosylated O-EPA.

Preferably, the purity of each of bioconjugate compositions (a)-(g) is at least 90%, e.g. 90%, 91%, 92%, 93%, 94%, 95%, 96%, 97%, 98%, 99%, 99.5%, 99.7%, 99.9% or 100%, as measured by SE-HPLC or at least 95%, e.g. 95%, 96%, 97%, 98%, 99%, 99.5%, 99.7%, 99.9% or 100% as measured by RP-HPLC. Typically, the purity of each of bioconjugate compositions (a)-(g) is at least 95%, e.g. at least 96%, 97%, 98%, 99%, as measured by either of SE-HPLC or RP-HPLC. In certain embodiments, one or more of bioconjugate compositions (a)-(g) are in containers and have a volume of between about 0.5 mL to about 100 L, e.g. about 1 mL to about 10 L, and the concentration of the bioconjugates is from about 4 μg/mL to about 5000 μg/mL, e.g. from about 15 μg/mL to about 1000 μg/mL, e.g. about 16 or 32 μg/mL (concentrations measured based on amounts of polysaccharide, as customary in this field; typically the polysac-charide/protein (w/w) ratio of these bioconjugates is about 0.1 to about 0.5, e.g. about 0.15 to about 0.45, such as about 0.2 to about 0.4.

In an embodiment, the pharmaceutical composition comprises bioconjugate composition (a).

In an embodiment, the pharmaceutical composition comprises bioconjugate composition (b).

In an embodiment, the pharmaceutical composition comprises bioconjugate composition (f).

In an embodiment, the pharmaceutical composition comprises at least four bioconjugate compositions chosen from (a)-(g) as defined above, preferably at least (a), (b), (c) and (f).

In another embodiment, the pharmaceutical composition comprises all seven bioconjugate compositions (a)-(g) as defined above.

In another embodiment, the pharmaceutical composition as defined above further comprises a bioconjugate composition (h) of O-EPA bioconjugate of E. coli serotype O15. In certain embodiments, O15-EPA bioconjugate composition (h) comprises a carrier protein having SEQ ID NO: 1 (EPA) to which O-antigen polysaccharide of E. coli serotype O15 is covalently attached.

The O15-EPA bioconjugate may be either mono-glycosylated with 1 O-antigen polysaccharide attached to EPA or multi-glycosylated with 2, 3 or 4 O-antigen polysaccharides attached to EPA, so that the bioconjugate composition is a mixture of mono-glycosylated and multi-glycosylated forms of the O15-EPA bioconjugate, wherein at least 50% is multi-glycosylated. In an embodiment, the bioconjugate composition (h) of O-EPA bioconjugate of E. coli serotype O15 comprises at least 50% multi-glycosylated O-EPA and not more than 95% multi-glycosylated O-EPA. Thus, in certain embodiments, bioconjugate composition (h) comprises between 50-95% multi-glycosylated O-EPA, e.g. 50%, 55%, 60%, 65%, 70%, 75%, 80%, 81%, 85%, 90% or 95% multi-glycosylated O-EPA. Furthermore, in certain embodiments, bioconjugate composition (h) comprises at least 5% mono-glycosylated O-EPA and not more than 50% mono-glycosylated O-EPA. Thus, in certain embodiments, bioconjugate composition (h) comprises between 5-50% mono-glycosylated O-EPA, e.g. 5%, 10%, 15%, 20%, 25%, 30%, 35%, 40%, 45%, or 50% mono-glycosylated O-EPA.

The percentages for multi-glycosylated and mono-glycosylated O-EPA are determined by cGE as described above.

Preferably, the purity of bioconjugate composition (h) is at least 90%, e.g. 90%, 91%, 92%, 93%, 94%, 95%, 96%, 97%, 98%, 99%, 99.5%, 99.7%, 99.9% or 100%, as measured by SE-HPLC or at least 95%, e.g. 95%, 96%, 97%, 98%, 99%, 99.5%, 99.7%, 99.9% or 100% as measured by RP-HPLC. Typically, the purity of bioconjugate composition (h) is at least 95%, e.g. at least 96%, 97%, 98%, 99%, as measured by either of SE-HPLC or RP-HPLC.

In certain embodiments, the pharmaceutical composition as described above comprises each of bioconjugate compositions (a), (b), (c), (d), (e), (f), (g), and (h) and has a volume of between about 0.5 mL to about 10 L, e.g. about 1 mL to about 1 L, and the total concentration of the bioconjugates is from about 90 μg/mL to about 600 μg/mL, e.g. from about 170 μg/mL to about 300 μg/mL, (measured based on amounts of polysac-charide, as customary in this field; typically the polysaccharide/protein (w/w) ratio of the bioconjugates in the composition is about 0.1 to about 0.5, e.g. about 0.15 to about 0.45, such as about 0.2 to about 0.4).

In another embodiment, the pharmaceutical composition as defined above further comprises a bioconjugate composition (i) of O-EPA bioconjugate of E. coli serotype O4.

In certain embodiments, O4-EPA bioconjugate composition (i) comprises a carrier protein having SEQ ID NO: 1 (EPA) to which O-antigen polysaccharide of E. coli serotype O4 is covalently attached.

The O4-EPA bioconjugate may be either mono-glycosylated with 1 O-antigen polysaccharide attached to EPA or multi-glycosylated with 2, 3 or 4 O-antigen polysaccharides attached to EPA, so that the bioconjugate composition is a mixture of mono-glycosylated and multi-glycosylated forms of the O4-EPA bioconjugate, wherein at least 10% is multi-glycosylated. The percentages for multi-glycosylated and mono-glycosylated O-EPA are determined by cGE as described above.

In an embodiment, the bioconjugate composition (i) of O-EPA bioconjugate of E. coli serotype O4 comprises at least 10% multi-glycosylated O-EPA and not more than 60% multi-glycosylated O-EPA. Thus, in certain embodiments, bioconjugate composition (i) comprises between 10-60% multi-glycosylated O-EPA, e.g. 10%, 15%, 20%, 25%, 30%, 35%, 40%, 44%, 45%, 50%, 55% or 60% multi-glycosylated O-EPA. Furthermore, in certain embodiments, bioconjugate composition (i) comprises at least 40% mono-glycosylated O-EPA and not more than 90% mono-glycosylated O-EPA. Thus, in certain embodiments, bioconjugate composition (i) comprises between 40-90% mono-glycosylated O-EPA, e.g. 40%, 45%, 50%, 55%, 60%, 65%, 70%, 75%, 80%, 85% or 90% mono-glycosylated O-EPA.

Preferably, the purity of bioconjugate composition (i) is at least 90%, e.g. 90%, 91%, 92%, 93%, 94%, 95%, 96%, 97%, 98%, 99%, 99.5%, 99.7%, 99.9% or 100%, as measured by SE-HPLC or at least 95%, e.g. 95%, 96%, 97%, 98%, 99%, 99.5%, 99.7%, 99.9% or 100% as measured by RP-HPLC. Typically, the purity of bioconjugate composition (i) is at least 95%, e.g. at least 96%, 97%, 98%, 99%, as measured by either of SE-HPLC or RP-HPLC.

In certain embodiments, the pharmaceutical composition as described above comprises each of bioconjugate compositions (a), (b), (c), (d), (e), (f), (g) and (i) or each of bioconjugate compositions (a), (b), (c), (d), (e), (f), (g), (h) and (i), and in each case has a volume of between about 0.5 mL to about 10 L, e.g. about 1 mL to about 1 L, and the total concentration of the bioconjugates is from about 90 μg/mL to about 600 μg/mL, e.g. from about 170 μg/mL to about 300 μg/mL, (measured based on amounts of polysaccharide, as customary in this field; typically the polysaccharide/protein (w/w) ratio of the bioconjugates in the composition is about 0.1 to about 0.5, e.g. about 0.15 to about 0.45, such as about 0.2 to about 0.4).

In certain embodiments, the pharmaceutical composition as defined above further comprises a bioconjugate composition (j) of O-EPA bioconjugate of E. coli serotype O8. In certain embodiments, the O8-EPA bioconjugate composition comprises O-EPA bioconjugate comprising a carrier protein having an amino acid sequence of SEQ ID NO: I (EPA) to which O-antigen polysaccharide of E. coli serotype O8 is covalently attached. Preferably, the purity of bioconjugate composition (j) is at least 90%, e.g. 90%, 91%, 92%, 93%, 94%, 95%, 96%, 97%, 98%, 99%, 99.5%, 99.7%, 99.9% or 100%, as measured by SE-HPLC or at least 95%, e.g. 95%, 96%, 97%, 98%, 99%, 99.5%, 99.7%, 99.9% or 100% as measured by RP-HPLC. Typically, the purity of bioconjugate compositions (j) is at least 95%, e.g. at least 96%, 97%, 98%, 99%, as measured by either of SE-HPLC or RP-HPLC. Thus, in certain embodiments, the pharmaceutical composition as described above comprises each of bioconjugate compositions (a), (b), (c), (d), (e), (f), (g), (h), (i) and (j) and has a volume of between about 0.5 mL to about L, e.g. about 1 mL to about 1 L, and the total concentration of the bioconjugates is from about 90 μg/mL to about 600 μg/mL, e.g. from about 170 μg/mL to about 300 μg/mL, (measured based on amounts of polysaccharide, as customary in this field; typically the polysaccharide/protein (w/w) ratio of the bioconjugates in the composition is about 0.1 to about 0.5, e.g. about 0.15 to about 0.45, such as about 0.2 to about 0.4).

In further embodiments, conjugates of different E. coli serotypes (i.e. O-antigen polysaccharides covalently coupled to carrier protein) may be added, e.g. to obtain a pharmaceutical composition comprising 10-20 conjugates, e.g. O-EPA conjugates. Such conjugates of different E. coli serotypes may also be bioconjugates or bioconjugate compositions, and may also have been purified according to processes described herein.

### DESCRIPTION OF THE SEQUENCES

SEQ ID NO: 1 (EPA carrier protein including 4 N-linked glycosylation
consensus sequences)
GSGGGDQNATGSGGGKLAEEAFDLWNECAKACVLDLKDGVRSSRMSVDPAIADTNGQGVLHYSMVLEG
GNDALKLAIDNALSITSQGLTIRLEGGVEPNKPVRYSYTRQARGSWSLNWLVPIGHEKPSNIKVFIHE
LNAGNQLSHMSPIYTIEMGDELLAKLARDATFFVRAHESNEMQPTLAISHAGVSVVMAQAQPRREKRW
SEWASGKVLCLLDPLDGVYNYLAQQRCNLDDTWEGKIYRVLAGNPAKHDLDIKDNNNSTPTVISHRLH
FPEGGSLAALTAHQACHLPLEAFTRHRQPRGWEQLEQCGYPVQRLVALYLAARLSWNQVDQVIRNALA
SPGSGGDLGEAIREQPEQARLALTLAAAESERFVRQGTGNDEAGAASADVVSLTCPVAKDQNRTKGEC
AGPADSGDALLERNYPTGAEFLGDGGDVSFSTRGTQNWTVERLLQAHRQLEERGYVFVGYHGTFLEAA
QSIVFGGVRARSQDLDAIWRGFYIAGDPALAYGYAQDQEPDARGRIRNGALLRVYVPRWSLPGFYRTG
LTLAAPEAAGEVERLIGHPLPLRLDAITGPEEEGGRVTILGWPLAERTVVIPSAIPTDPRNVGGDLDP
SSIPDKEQAISALPDYASQPGKPPREDLKLGSGGGDQNAT
SEQ ID NO: 2 (example PglB oligosaccharyl transferase)
MLKKEYLKNPYLVLFAMIILAYVFSVFCRFYWVWWASEFNEYFFNNQLMIISNDGYAFAEGARDMIAG
FEQPNDLSYYGSSLSALTYWLYKITPFSFESIILYMSTFLSSLVVIPTILLANEYKRPLMGFVAALLA
SIANSYYNRTMSGYYDTQMLVIVLPMFILFFMVRMILKKDFFSLIALPLFIGIYLWWYPSSYTLNVAL
IGLFLIYTLIFHRKEKIFYIAVILSSLTLSNIAWFYQSAIIVILFALFALEQKRLNFMIIGILGSATL
IFLILSGGVDPILYQLKFYIFRSDESANLTQGFMYFNVNQTIQEVENVDLSEFMRRISGSEIVFLFSL
FGFVWLLRKHKSMIMALPILVLGFLALKGGLRFTIYSVPVMALGFGFLLSEFKAIMVKKYSQLTSNVC
IVFATILTLAPVFIHIYNYKAPTVFSQNEASLLNQLKNIANREDYVVTWWDYGYPVRYYSDVKTLVDG
GKHLGKDNFFPSFALSKDEQAAANMARLSVEYTEKSFYAPQNDILKTDILQAMMKDYNQSNVDLFLAS
LSKPDFKIDTPKTRDIYLYMPARMSLIFSTVASFSFINLDTGVLDKPFTFSTAYPLDVKNGEIYLSNG
VVLSDDFRSFKIGDNVVSVNSIVEINSIKQGEYKITPIDDKAQFYIFYLKDSAIPYAQFILMDKTMFN
SAYVQMFFLGNYDKNLFDLVINSRDAKVFKLKI
SEQ ID NO: 3 (example signal sequence for EPA carrier protein)
MKKIWLALAG LVLAFSASA

### Exemplary Glycosylation Consensus Sequence

Asn-X-Ser(Thr), wherein X can be any amino acid except Pro.

### Exemplary Optimised Glycosylation Consensus Sequence

Asp(Glu)-X-Asn-Z-Ser(Thr), wherein X and Z are independently selected from any amino acid except Pro.

The following examples of the invention are to further illustrate the nature of the invention. It should be understood that the following examples do not limit the invention and the scope of the invention is to be determined by the appended claims.

### Example 1: Novel Production Process for O-EPA Bioconjugates

This is an example of the inventive production process for ExPEC O-EPA bioconjugates.

### Bioconjugate Production Strains

Construction of production strains for the different bioconjugates has been previously described, e.g. in example 6 of WO2020/191082. For some bioconjugates the previously described strains were used, while for some other bioconjugates new production strains were constructed to improve yields. The parent cell is E. coli strain W3110. In the production strains, the rfb locus encoding the enzymes that are responsible for making the relevant E. coli O-antigen polysaccharide (serotype-specific, sequences of the respective rfb loci provided herein in Table 2 by reference to the sequences provided in WO 2020/191082 and WO WO 2022/208430) replaces the deleted endogenous W3110 rfb locus; the same EPA carrier protein (having 4 N-linked glycosylation sequences; the carrier protein having SEQ ID NO: 1) is encoded on a plasmid in each of these production strains; and the oligosaccharyl transferase that transfers the O-antigens to an Asn in the N-linked glycosylation sequences of the carrier protein is also encoded on a plasmid in these production strains and is a C. jejuni PglB having SEQ ID NO: 2 or a variant thereof that improves yields for specific E. coli O-antigen bioconjugates and that differs from SEQ ID NO: 2 by the mutations indicated in the last column of Table 2 below.

TABLE 2
Relevant features of production strains for ExPEC9V bioconjugates.

| Bioconjugate | | PglB variant |
|---|---|---|
| production | | (mutations |
| for | | compared |
| E. coli | | to SEQ ID |
| serotype | rfb cluster sequence | NO: 2) |
| O1A | SEQ ID NO: 11 of WO 2020/191082 | N311V, K482R, |
| | | D483H, A669V |
| O2 | SEQ ID NO: 12 of WO 2020/191082 | N534Q |
| O4 | SEQ ID NO: 9 of WO 2020/191082 | Y77H, N311V |
| O6A | SEQ ID NO: 13 of WO 2020/191082 | Y77H, S80R, |
| | | Q287P, K289R, |
| | | N311V |
| O15 | SEQ ID NO: 15 of WO 2020/191082 | N311V, K482R, |
| | | D483H, A669V |
| O16 | SEQ ID NO: 16 of WO 2020/191082 | Y77H, S80R, |
| | | Q287P, K289R, |
| | | N311V |
| O18A | SEQ ID NO: 5 of WO 2022/208430 | no mutations |
| | with mutations T199I, M377K, | |
| | and V395A in the Wzy protein | |
| | encoded by the way gene in said | |
| | rfb cluster | |
| O25B | SEQ ID NO: 18 of WO 2020/191082 | no mutations |
| O75 | SEQ ID NO: 19 of WO 2020/191082 | Y77R, N311V |

Note: In the strain for production of E. coli O4 bioconjugates, nucleic acid encoding the E. coli O4-specific GtrS protein (see, e.g., SEQ ID NOs: 4 and 5 of WO 2020/191082 for the amino acid sequence of this GtrS protein and a nucleic acid sequence encoding this protein, respectively) is also present in the genome to allow production of bioconjugates with Glc-branched O4 polysaccharides as shown in structure (O4) in Table 1 (see also WO 2020/191082).

The strains were used for the production of the bioconjugates by the inventive process described herein.

The inventive process as implemented in this example consists of the following steps: AEX1 (Capture AEX chromatography); particle reduction filtration 1; mixed-mode chromatography; buffer adjustment for HIC; particle reduction filtration 2; HIC; TFF1; particle reduction filtration 3; AEX2 (polishing chromatography); TFF2; bioburden filtration.

Buffers, including their composition, referred to within this and the following example are listed in Table 3 below.

### Step i: Providing a Filtered Periplasmic Fraction Comprising O-EPA Bioconjugate.

A filtered periplasmic fraction comprising the O25B-EPA bioconjugate served as the starting material for the purification process outlined below. Production of O-EPA bioconjugates es has been previously described (see e.g. WO2009/104074, WO2015/124769 and WO2020/191082) and was performed in analogy to these proto-cols. Specifically, the periplasmic fraction of E. coli host cells comprising an O25B-EPA bioconjugate was obtained by osmotic shock treatment after incubation in a 200 L bioreactor (fermenter) as previously described [WO2020191082]. A filtered periplasmic fraction was obtained, essentially as follows. Incubation in a 200 L bioreactor corresponds to 165 L harvest equivalent (HE; LHE).

In more detail:

As a harvest step, 165 L culture broth is cooled down to below 20° C., before the harvest by a disc stack centrifuge (DSC) is started, using a separator with a constant flow (e.g. about 160 L/h). The biomass is collected and the centrate (supernatant) is discarded.

The following step is the osmotic shock, which is performed using solutions at about 6-10° C. The cell wet weight (CWW) for the concentrated harvest is determined and based on the CWW the cell suspension is diluted to a target CWW of for instance about 390 g/L with 1/3 TBS (pH 7.4), and the total volume is determined. 60% sucrose solution is added to the diluted harvest to a target concentration of 25% and incubated at 6-10° C. for 1 h with the cells, while mixing the solution. After the incubation with sucrose the cell/sucrose solution is mixed in-line with 4× the volume of 10 mM Tris-HCL (pH 8.0), at 6-10° C. Mixing is performed through a static mixer. The product is released from the periplasmic space into the supernatant (called periplasmic fraction, PF), and the PF is collected.

Subsequently the material is clarified: the cell debris in the PF is removed by separation in a disc stack centrifuge. A constant flow is applied and based on the centrate turbidity the flow is adjusted. The supernatant is collected, and is referred to as centrifuged periplasmic fraction (CPF).

The CPF still contains cell debris and is filtered through a depth and bioburden reduction filter, and collected as filtered periplasmic fraction (FPF). The FPF was used for further purification of the O-EPA starting with the AEX1 step (step ii), as described further below.

### Step ii: Capture AEX1 Chromatography

ii-1: Adjusting the conductivity of filtered fermenter harvest was not required. The periplasmic fraction of osmotically shocked and filtered fermenter harvest was loaded in bind-elute mode on a Q Ceramic HyperD F resin for a first Anion Exchange Chromatography (AEX1; 20 cm bed height, 0.34 L resin/L harvest equivalent). After equilibration of the column with the low salt buffer A, the filtered periplasmic fraction comprising the O-EPA bioconjugate was loaded onto the column. The column was then washed with 3 column volumes (CV) of buffer A.

ii-2: Elution was achieved using 20% buffer BV2 in buffer A in a step gradient. The eluate was fractionated in 4 fractions of 0.25 CV, wherein collection of eluate fractions started when the UV-absorption increased with a slope greater than 1.0 AU/min.

ii-3: The “AEX1 pool” (AEX1 eluate) was created by mixing fractions 1 and 2.

Step iii: Mixed-mode chromatography

iii-1: Particle reduction filtration was performed using a PES membrane (Sartopore) with 0.45+0.2 μm cut-off and 0.45 m²/165 LHE filter area.

iii-2: Mixed-mode chromatography (Capto adhere multimodal resin, 20 cm bed height, 6.3 L/165 LHE harvest equivalent) was performed in bind-elute mode.

After pre-equilibration with 3 CV buffer CC, the column was equilibrated with 5 CV buffer AA and the AEX1 eluate was applied on a Capto Adhere multimodal resin (bind-elute mode). The column was washed using 5 CV buffer AA, then 5 CV buffer BB and finally 2 CV buffer AA.

iii-3: Elution was performed using 3.5 CV buffer CC. The eluate was collected after UV slope (measured at 280 nm) exceeded 0.05 mAU (MMR eluate).

iii-4: Two fractions of 1 CV each were collected and pooled without prior analysis by SDS-PAGE.

Step iv: HIC chromatography

iv-1: To the pooled MMR eluate was added adjustment buffer (buffer Q) until the added weight reached 3 times the initial weight of the pooled MMR eluate.

iv-2: Particle reduction filtration was performed using a PES membrane (Sartopore) with 0.45+0.2 μm cut-off and 0.45 m^{2/165} LHE filter area.

iv-3: HIC was performed in bind-elute mode. The HIC capsule (Sartobind Phenyl Jumbo 5 L, 0.8 cm bed height) was equilibrated with 3 CV Buffer Q and the adjusted load of the MMR eluate was applied. Then, the HIC capsule was washed with 1 CV Buffer Q, followed by 4 CV of a buffer mixture containing 70% Buffer Q and 30% Buffer R.

iv-4: The O-EPA bioconjugate was eluted by applying 4 CV of a buffer mixture containing 30% Buffer Q and 70% Buffer R. The eluate was fractionated in 4 fractions of 0.8 CV. Collection of eluate fractions started when the UV-absorption increased with a slope greater than 0.5 AU/min and the UV-adsorption value was greater than 0.05 AU.

iv-5: The “HIC pool” was created by mixing fractions 1-3 without prior analysis by SDS-PAGE.

### Step v: Polishing AEX2 Chromatography

v-1: A tangential flow filtration (TFF1) was conducted in order to condition the HIC eluate for the polishing step, i.e. AEX2. The HIC eluate was diafiltered by TFF (mPES KrosFlo Filter Module Q, 10 kDa, 1.25 m^{2/165} LHE, trans membrane pressure ca. 0.8 bar) with buffer U until reaching the target diafiltration volume of 5 to 6 DV.

v-2: The adjusted HIC eluate was then filtered using a Sartopore 2 Capsule Size O with a PES membrane with 0.45+0.2 μm cut-off and 0.45 m² filter area.

v-3: The polishing AEX chromatography (AEX2) was performed in bind-elute mode. The column (Source 15Q, 20 cm bed height, 6.3 L/165 LHE) was equilibrated using 3 CV Buffer U. The adjusted HIC eluate was applied on the column.

Elution was performed using first 21% Buffer V in Buffer U for 7.5 CV (step gradient), then a linear gradient of 21-56% Buffer V in Buffer U over 7.5 CV. Fractionation was started after 1.2 CV of the Elution start and subsequently 30 fractions of 0.5 CV were collected.

v-4: The individual fractions were analyzed by SDS-PAGE and stained by Coomassie staining. The AEX2 eluate pool was created by mixing fractions starting from the first fraction showing a strong product band together with only weak to medium intensity impurity bands until the first fraction showing fading product bands. Fractions showing fading product bands often show additional bands of increasing intensity corresponding to unglycosylated-EPA. In this case, fractions 11-30 were pooled. In this example, the O25B-EPA bioconjugate was obtained with a purity of 99.4% as measured by SE-HPLC.

### Step vi: Adjustment to a Pharmaceutically Acceptable Buffer and Concentration

vi-1: A second TFF (mPES KrosFlo Filter Module Q, 10 kDa, 1.25 m²/165 LHE) was applied to obtain the O-EPA bioconjugate at a concentration of OD280=0.5=0.1 using an excipient buffer comprising 6.19 mM KH₂PO₄, 3.81 mM Na₂HPO₄, 5% (w/w) sorbitol, 10 mM methionine, at pH 7.0 (Buffer XV2). The AEX2 eluate was then dia-filtered with the excipient buffer over 5-6 diafiltration volumes, and then concentrated to a target concentration of OD280=1.40±0.15. Then, polysorbate-80 (Tween-80) was added to the excipient buffer to a final concentration of 0.02% (w/w) to obtain the O25B-EPA conjugate in a pharmaceutically acceptable buffer (Tween-adjusted Buffer XV2) containing 6.19 mM KH₂PO₄, 3.81 mM Na₂HPO₄, 5% (w/w) sorbitol, 10 mM methionine, 0.02% (w/w) polysorbate-80, at pH 7.0 (see e.g. WO 2018/077853).

vi-2: A bioburden filtration was applied using a Sartopore 2 Capsule Size 9 with a PES membrane (0.45+0.2 μm cut-off and 0.2 m² filter area).

vi-3: The resulting product (drug substance) was filled into bottles (drug substance bulk) and frozen at −70° C.+/−10° C. in a primary freezer. After 72 h DS bottles were removed from the primary freezer and directly placed into a final storage freezer at −70° C.+/−10° C. without allowing any thawing of the DS in between.

In this example, the O25B-EPA bioconjugate was obtained with a purity of 99.6% as measured by SE-HPLC, and a yield of 24 mg PS/L HE, corresponding to an estimated overall yield of about 23% (relative to O-EPA conjugate present in the FPF of step i above).

A broad range of bioconjugates were purified using the same inventive process as described above. In particular, O-EPA bioconjugates were purified for E. coli serotypes O1A, O2, O4, O6A, O15, O16, O18A, O25B, and O75. The purity for each of these was typically at least 96% as measured by SE-HPLC, and in most cases purity was 98-100% as measured by SE-HPLC. The estimated overall yields of the process differed per strain and were inter alia dependent on starting expression levels, and varied between about 5-35%, with a mean of about 20%. This demonstrates that the process of the invention is widely applicable for various different O-EPA conjugates, and is suitable for economic large scale production of any O-EPA conjugate to very high purity, which is sufficient for administration of the O-EPA conjugate to humans.

The E. coli O-antigen bioconjugates thus prepared that are drug substances (DSs) showed comparable critical quality attributes:
(1) purity (measured by SE-HPLC or RP-HPLC) was higher than 95%.
(2) polysaccharide/protein ratio ranged between about 0.1-0.5, mostly between 0.15 and 0.45.
(3) bacterial endotoxin (Ph. Eur. 2.2.3) was less than 0.5 EU/ug polysaccharide.
(4) The average length of the individual polysaccharide chains was typically between about 10-20 repeating units (measured using high resolution SDS-PAGE).

### Example 2 (Comparative): Process According to WO 2022/214620

This is a comparative example of the previously preferred production process for ExPEC O-EPA bioconjugates (see WO 2022/214620). Only differences to the inventive process outlined above are described. The most prominent change in the inventive process of the present invention compared to the previously described process (e.g. WO 2022/214620) concerns the replacement of the hydroxyapatite step by a mixed-mode chromatography step (step iii).

### Step iii: Hydroxyapatite Chromatography

iii-1: The pooled AEX1 fractions were adjusted to a tar-get pH of 7.2=0.2 using Buffer Y and in a subsequent step to a target conductivity of 7.5-9 mS/cm using buffer Z.

iii-2: Particle reduction filtration was performed as described for the inventive process above.

iii-3: Ceramic hydroxyapatite chromatography (cHA, 20 cm bed height, 0.34 L resin/L harvest equivalent) was performed in bind-elute mode.

After pre-equilibration with 6 CV Buffer N to adjust pH followed by equilibration with 6 CV Buffer J, the ad-justed load of the AEX1 eluate was applied onto the cHA resin. The column was washed using 1 CV of a low conductivity buffer consisting of 5% buffer K in buffer J.

iii-4: Elution of the product was performed using a salt gradient, i.e. a sodium chloride and potassium phosphate gradient. At first a linear gradient from 20-45% Buffer K in Buffer J over 2.5 CV was applied, followed by a step gradient to 70% Buffer K in Buffer J for 2.5 CV. Collection of 14 fractions of 15.565 L/165L HE was started after 1.9 CV of the linear gradient had been applied.

iii-5: Individual fractions were analyzed by SDS-PAGE. Selection of fractions for cre-ating a “cHA pool” was based on the intensity of the band corresponding to the O-EPA bioconjugate relative to impurities. The pooled cHA eluate is then created by mixing the selected fractions.

### Step iv: HIC

iv-1: In this previous process, a load of the eluate from the previous step was adjusted until a target conductivity of 118±2 mS/cm was reached. In the inventive process, adjustment buffer is added until the added weight reaches 3 times the initial weight of the pooled eluate of the previous chromatography step. Hence, this step has been sim-plified in the inventive process.

### Step v: Polishing AEX2 Chromatography

v-1: A tangential flow filtration (TFF1) is conducted in order to condition the HIC eluate for the polishing step, i.e. AEX2. The HIC eluate first is adjusted (diluted or concentrated) by TFF (mPES KrosFlo Filter Module Q, 10 kDa, 1.25 m^{2/165} LHE, trans membrane pressure ca. 0.8 bar) with Buffer T to an OD280 of 1.35=0.15 and then dia-filtered with buffer T to reach the target conductivity of 5-6 mS/cm. In some cases, pH of the HIC eluate needs additional adjustment prior to the subsequent chromatography step. In this case, pH is decreased to a target pH of 6.5±0.2 using buffer Y. In a further step, Buffer V was used to adjust the conductivity to 8.7±0.3 mS/cm. Thus, adjustment of the HIC eluate includes at least two steps (adjustment of concentration and diafiltration), in some cases three steps (including pH adjustment). In contrast, for the inventive process (example 1), adjustment of the HIC eluate includes only one step (diafiltration).

v-3: The adjusted HIC eluate was applied on the column (same column as example 1) and a wash step was performed using 1.5 CV Buffer U. In a second wash, a linear gradient of 15-21% Buffer V in Buffer U was applied. In contrast, for the inventive process (example 1), a wash step is not required after applying the HIC eluate on the AEX2 column.

v-4: Elution was performed using first 21% Buffer V in Buffer U for 3 CV (step gradient), then a linear gradient of 21-56% Buffer V in Buffer U over 7.5 CV. Fractionation was started after 1.25 CV of the Elution start and subsequently 20 fractions of 0.4975 CV were collected. Thus, the step gradient is extended in the inventive process compared to this previous process (7.5 CV instead of 3 CV).

v-5: The individual fractions are analyzed by SDS-PAGE and stained by Coomassie staining. The AEX2 eluate pool is created by mixing the first fraction with an OD280 of at least 0.04 AU. Thus, in this comparative process pooling of fractions is based on an OD280 value of at least 0.04 AU whereas in the inventive process (example 1), OD measurement is not required. Instead pooling of fractions is based on SDS-PAGE analysis only.

In this example, the O25B-EPA bioconjugate was obtained with a purity of 99.7% as measured by SE-HPLC.

### Step vi: TFF2

vi-1: In contrast to the inventive process (example 1), adjusting the pH of a load of the AEX2 eluate prior to TFF2 is required in this comparative process (only optional and typically not performed in the inventive process).

TABLE 3
Buffers

| Name | Buffer composition |
|---|---|
| Buffer A | 10 mM Tris, 50 mM NaCl, pH 8.5 |
| Buffer AA | 50 mM BisTris, 50 mM NaCl, pH 6.0 |
| Buffer BV2 | 50 mM Tris, 1M NaCl, pH 8.5 |
| Buffer BB | 50 mM acetate, 250 mM NaCl, pH 4.7 |
| Buffer CC | 50 mM BisTris, 2M NaCl, pH 6.0 |
| Buffer F | 1M NaOH |
| Buffer FV2 | 0.495M NaOH |
| Buffer GV2 | 1M Acetic acid |
| Buffer IV2 | 20% EtOH |
| Buffer J | 30 mM BisTris, 2.5 mM K-phosphate, |
| | 50 mM NaCl, pH 7.0 |
| Buffer K | 30 mM BisTris, 32 mM K-phosphate, |
| | 450 mM NaCl, pH 7.0 |
| Buffer 5% | 30 mM BisTris, 3.975 mM K-phosphate, |
| K in J | 70 mM NaCl, pH 7.0 |
| Buffer L | 400 mM K-phosphate, pH 7.0 |
| Buffer M | 0.1M NaOH |
| Buffer N | 100 mM BisTris, 2.5 mM K-phosphate, |
| | 50 mM NaCl, pH 7.2 |
| Buffer O | 30 mM BisTris, 50 mM CaCl₂, pH 7.2 |
| Buffer P | 30 mM BisTris, pH 7 |
| Buffer Q | 2M K-phosphate, pH 7.0 |
| Buffer R | WFI water |
| Buffer T | 10 mM BisTris, pH 6.0 |
| Buffer U | 10 mM BisTris, 50 mM NaCl, pH 6.0 |
| Buffer V | 10 mM BisTris, 200 mM NaCl, pH 6.0 |
| Buffer W | 100 mM Na₂HPO₄ |
| Buffer XV2 | 10 mM Phosphate, 5% (w/w) Sorbitol, 1 |
| | 0 mM Methionine, pH 7.0 |
| Buffer Y | 0.5M BisTris, pH 6.0 |
| Buffer Z | 10 mM BisTris, pH 7.0 |
| 10% (w/w) | 10% (w/w) SUPER REFINED |
| Tween-80 in | POLYSORBATE 80 in Buffer XV2 |
| Buffer XV2 | |
| Culture medium | 330 mM Glycerol, 10 g Yeast |
| | extract, 20 g Tryptone, 34 mM |
| | K₂HPO₄, 22 mM KH₂PO₄, |
| | 38 mM (NH₄)₂SO₄, 2 mM |
| | MgSO₄•7 H₂O and 5 mM citric acid |
| Suspension | 25% Sucrose, 100 mM EDTA, 200 mM |
| buffer | Tris HCl pH 8.5 |
| Tween-adjusted | 6.19 mM KH₂PO₄, 3.81 mM |
| Buffer XV2 | Na₂HPO₄, 5% (w/w) sorbitol, 10 |
| | mM methionine, 0.02% (w/w) |
| | polysorbate-80, at pH 7.0 |

### Example 3: Increased Multi-Glycosylation of O-EPA Bioconjugates Obtained by Inventive Process

This example describes the degree of glycosylation (DOGY) of three O-EPA bioconjugate compositions (E. coli O-antigen polysaccharides corresponding to serotypes O1A, O2 and O25B, each individually coupled to EPA carrier protein having SEQ ID NO: 1) produced by the inventive method (example 1) and the comparative method according to WO 2022/214620 (example 2).

For each pair of the O-EPA bioconjugates shown in Table 4, the same production strain (features indicated in Table 2 above) and fermentation conditions were used.

DOGY was determined by cGE according to the following protocol:

O-EPA bioconjugates were concentrated to a target concentration of 3 mg/mL and separated in a bare-fused silica capillary (total capillary length of 30.2 cm and an inner diameter of 50 μm) containing a SDS-Gel buffer (pH 8.0, 0.2% SDS, 1% PEG6000). Sample separation was done at −15 kV and absorbance of the bioconjugate was measured at 220 nm. The relative amount of mono-, di-, tri- and tetra-glycosylated forms is determined by integration of the corresponding peak sections.

For each measurement, a respective O-EPA bioconjugate drug substance from a previous production batch is used as a reference material and analysed under the same conditions to ensure correct performance of the method (external control).

TABLE 4
Degree of glycosylation of O-EPA bioconjugate compositions
comprising O-antigen polysaccharide of E. coli serotypes
O1A, O2, or O25B individually coupled to EPA carrier
protein having SEQ ID NO: 1, as measured by cGE and
produced according to this invention (example 1;
“inventive”) compared to the prior art (example 2; “comparative”).

| | | Mono- | Di- | Tri- | Total |
|---|---|---|---|---|---|
| Bioconjugate | | glyco- | glyco- | glyco- | multi- |
| composition | Process | sylated | sylated | sylated | glycosylated |
| O1A | comparative | 55% | 39% | 7% | 46% |
| | inventive | 33% | 52% | 15% | 67% |
| O2 | comparative | 59% | 41% | 0% | 41% |
| | inventive | 29% | 71% | 0% | 71% |
| O25B | comparative | 59% | 41% | 0% | 41% |
| | inventive | 34% | 66% | 0% | 66% |

For each of these three different bioconjugates, the production strains and fermentation conditions were identical between the process according to WO 2022/214620 (comparative) and the inventive process described herein and any differences in the obtained products could thus be attributed to the differences in the purification process. The inventive process of the instant invention surprisingly led to less mono-glycosylated and more multi-glycosylated O-EPA bioconjugate in the final purified product as compared to the comparative and previously preferred process as described in WO 2022/214620. This increase in multi-glycosylation in the bioconjugate compositions is advantageous in that more of the available glycosylation sites in the EPA carrier protein appear used in the product thus obtained, leading to potentially more available immunogenic glycans per carrier protein molecule.

In addition to the improvement in percentages of multi-glycosylated O-EPA, the new process of the invention (see example 1) for several serotypes generally led to even lower levels of transaldolase B (the most abundant residual host cell proteins) in the purified O-EPA bioconjugate compositions, as compared to the transaldolase B levels obtained with the comparative process (i.e. having a cHA step rather than MMC step as second chromatography step; see example 2).

Furthermore, for several serotypes, the novel process of the invention described above (example 1) led to a significant reduction in buffer consumption, typically about 81% reduction in buffer consumption for operating the 2nd column, as compared to the previously preferred process (example 2).

### Example 4: Degree of Glycosylation of 9 O-EPA Bioconjugate Compositions

This example describes the degree of glycosylation (DOGY) of several O-EPA bioconjugate compositions (E. coli O-antigen polysaccharides corresponding to serotypes O1A, O2, O4, O6A, O15, O16, O18A, O25B and O75, each individually coupled to EPA carrier protein having SEQ ID NO: 1) produced by the inventive method (example 1). Since the bioconjugate production strain for an individual bioconjugate could also have an effect on DOGY, e.g. depending on the genes in the rfb locus and/or the PglB variant used, these relevant characteristics of the production strains are identified in Table 2 above. Several batches were produced for each bioconjugate composition with some DOGY variation. Results obtained from material that was produced in the context of a production campaign for clinical trials are shown in Table 5. DOGY was measured by cGE as described in example 3.

TABLE 5
Degree of glycosylation of O-EPA bioconjugate compositions
comprising O-antigen polysaccharide of E. coli serotypes
O1A, O2, 04, O6A, O15, O16, O18A, O25B, or O75 individually
coupled to EPA carrier protein having SEQ ID NO: 1, as measured by
cGE and produced from production strains according to Table 2 and
according to the inventive method described herein (example 1).

| | | | | Total |
|---|---|---|---|---|
| Bioconjugate | Mono- | Di- | Tri- | multi- |
| composition | glycosylated | glycosylated | glycosylated | glycosylated |
| O1A | 33% | 52% | 15% | 67% |
| O2 | 29% | 71% | 0% | 71% |
| O4 | 57% | 40% | 4% | 44% |
| O6A | 27% | 46% | 26% | 72% |
| O15 | 19% | 57% | 24% | 81% |
| O16 | 24% | 47% | 29% | 76% |
| O18A | 12% | 49% | 39% | 88% |
| O25B | 34% | 66% | 0% | 66% |
| O75 | 31% | 48% | 22% | 70% |

The sum of mono-, di- and tri-glycosylated O-EPA bioconjugate compositions may slightly deviate from 100% due to rounding effects.

For O4 there was a decrease in multi-glycosylated over mono-glycosylated O-EPA in compositions so obtained versus compositions obtained using a previous production strain (WO 2020/191082) and the previous production process (example 2). For O15, fraction of multi-glycosylated forms was comparable in compositions so obtained versus compositions obtained using a previous production strain (WO 2020/191082) and the previous production process (example 2). However, the other of these bioconjugate compositions have multi-glycosylation levels that had not previously been described and were not obtained in bioconjugates of the same O-serotype coupled to the same carrier protein but prepared according to processes of the prior art, i.e. in most cases the novel bioconjugates of the invention have increased levels of multi-glycosylation.

### Example 5: Immunogenicity of 9-Valent Drug Product in Rabbits

Nine monovalent drug substances, each corresponding to one of O-EPA bioconjugates comprising EPA carrier protein having SEQ ID NO: 1 coupled to O-antigen polysac-charide of E. coli serotypes O1A, O2, O4, O6A, O15, O16, O18A, O25B, or O75, and obtained according to the inventive process described herein (example 1; production strains identified in Table 2), were mixed (step (vii) according to the invention, counting further from example 1) to obtain a 9-valent drug product composition (see e.g. WO 2022/058945), which was suitable for use in humans.

As outlined above, the new process for several serotypes generally led to even lower levels of transaldolase B in the purified O-EPA bioconjugate compositions, as compared to the transaldolase B levels obtained with the comparative process (i.e. having a cHA step rather than MMC step as second chromatography step; see example 2). Overall, for the 9-valent drug product composition prepared using the novel process according to the invention, the remaining amount of transaldolase B impurity was further reduced by a factor of about 2-3 as compared to a 10-valent drug product composition that includes the same dose of polysaccharide prepared using the comparative process. This lower level of this impurity is advantageous for a pharmaceutical product.

The 9-valent drug product composition prepared using the novel process of the invention was administered to rabbits and induced antibodies against the E. coli serotypes of which O-antigens were present in the composition (O1A, O2, O4, O6A, O15, O16, O18A, O25B, and O75). Thus, the bioconjugates and compositions (obtained) according to (methods of) the invention were demonstrated to be suitable to induce immune responses against E. coli.

1. A pharmaceutical composition comprising one or more bioconjugate compositions,
wherein each bioconjugate composition comprises a Pseudomonas aeruginosa ExoProtein A carrier protein (EPA) having SEQ ID NO: 1 to which O-antigen polysaccharide of a specific E. coli serotype is covalently attached (O-EPA bioconjugate),
wherein the O-EPA bioconjugates may be either mono-glycosylated with 1 O-antigen polysaccharide attached to EPA or multi-glycosylated with 2, 3 or 4 O-antigen polysaccharides attached to EPA, so that each of said one or more bioconjugate compositions is a mixture of mono-glycosylated and multi-glycosylated forms of the O-EPA bioconjugate, characterized in that the pharmaceutical composition comprises one or more of the following:
(a) a bioconjugate composition of O-EPA bioconjugate of E. coli serotype O1A, wherein at least 50% is multi-glycosylated;
(b) a bioconjugate composition of O-EPA bioconjugate of E. coli serotype O2, wherein at least 44% is multi-glycosylated;
(c) a bioconjugate composition of O-EPA bioconjugate of E. coli serotype O6A, wherein at least 64% is multi-glycosylated;
(d) a bioconjugate composition of O-EPA bioconjugate of E. coli serotype O16, wherein at least 65% is multi-glycosylated;
(e) a bioconjugate composition of O-EPA bioconjugate of E. coli serotype O18A, wherein at least 70% is multi-glycosylated;
(f) a bioconjugate composition of O-EPA bioconjugate of E. coli serotype O25B, wherein at least 50% is multi-glycosylated; and/or
(g) a bioconjugate composition of O-EPA bioconjugate of E. coli serotype O75, wherein at least 50% is multi-glycosylated,
wherein each of the percentages for multi-glycosylated and mono-glycosylated O-EPA are determined by capillary gel electrophoresis.

2. The pharmaceutical composition of claim 1 comprising at least one of bioconjugate compositions (a), (b) and (f).

3. The pharmaceutical composition according to claim 1, comprising all seven bioconjugate compositions (a)-(g).

4. The pharmaceutical composition according to claim 2, further comprising:
(h) a bioconjugate composition of O-EPA bioconjugate of E. coli serotype O15 and (i) a bioconjugate composition of O-EPA bioconjugate of E. coli serotype O4; or
further comprising:
(h) a bioconjugate composition of O-EPA bioconjugate of E. coli serotype O15, (i) a bioconjugate composition of O-EPA bioconjugate of E. coli serotype O4 and (j) a bioconjugate composition of O-EPA bioconjugate of E. coli serotype O8.

5. A process for production of a purified bioconjugate from Gram-negative bacterial host cells wherein the bioconjugate comprises a bacterial O-antigen polysaccharide covalently coupled to a Pseudomonas aeruginosa ExoProtein A carrier protein (O-EPA), the process comprising:
i. providing a filtered periplasmic fraction (FPF) of the host cells that express the bioconjugate, the FPF comprising the bioconjugate;
ii. subjecting an adjusted load of the FPF to a first anion exchange chromatography (AEX 1) step to obtain a first AEX eluate (AEX1) that comprises the bioconjugate;
iii. subjecting an adjusted load of the AEX1 eluate to a mixed-mode chromatography (MMC) step on a multimodal resin (MMR) to obtain an MMR eluate that comprises the bioconjugate, said MMR comprising both anion exchange functionalities and hydrophobic functionalities;
iv. subjecting an adjusted load of the MMR eluate to a hydrophobic interaction chromatography (HIC) step to obtain a HIC eluate comprising the bioconjugate; and
v. subjecting an adjusted load of the HIC eluate to a second anion exchange chromatography (AEX 2) step to obtain an AEX2 eluate comprising the bioconjugate, wherein in purification steps (ii) to (v) conditions are first adjusted to allow binding of the bioconjugate to a chromatography medium and subsequently adjusted to allow elution of the bioconjugate from said chromatography medium.

6. The process according to claim 5, wherein the MMR comprises ligands of formula (I), 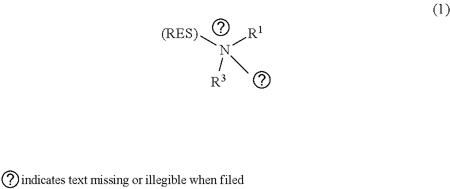
wherein
RES represents the resin of the MMR;
R¹ represents C₁-C₄ alkyl;
R² represents C₁-C₄ alkyl substituted with phenyl, tolyl or xylyl;
R³ represents C₁-C₄ alkyl substituted with a hydroxyl or thiol group.

7. The process according to claim 6, wherein the ligand of formula (I) is N-benzyl-N-methylethanolamine.

8. The process according to claim 5, wherein the O-polysaccharide is specific to a Gram-negative bacterium selected from the list of Escherichia and Shigella.

9. The process according to claim 5, wherein the Gram-negative bacterial host cells from which the bioconjugate is obtained comprise genetic information encoding:
(a) a bacterial O-antigen polysaccharide;
(b) a recombinant Pseudomonas aeruginosa ExoProtein A protein (EPA) that comprises at least one glycosylation site; and
(c) a metabolic apparatus that carries out N-glycosylation of the EPA with the O-antigen polysaccharide,
thereby producing the bioconjugate in vivo in the periplasm of the host cells.

10. The process according to claim 9, wherein the recombinant EPA comprises 4 N-glycosylation sites having the amino acid sequence Asp(Glu)-X-Asn-Z-Ser(Thr), wherein X and Z are independently selected from any amino acid except Pro.

11. The process according to claim 10, wherein the recombinant EPA comprises the amino acid sequence of SEQ ID NO: 1.

12. The process according to claim 5, wherein the bacterial O-antigen polysaccharide is an E. coli O-antigen polysaccharide.

13. The process according to claim 5, wherein the host cells have been cultured in a bioreactor with a volume of between 100 L and 20000 L.

14. The process according to claim 5, wherein in an additional step (vi) a load of the AEX2 eluate is adjusted to a pharmaceutically acceptable buffer and concentration thereby obtaining the purified O-EPA conjugate as a pharmaceutical drug substance.

15. The process according to claim 14, wherein in an additional step (vii) several purified bioconjugate drug substances are combined thereby obtaining a multivalent drug product,
wherein the multivalent drug product comprises at least four O-antigen polysaccharides,
and wherein said O-antigen polysaccharides are selected from the group consisting of E. coli O-antigen polysaccharide O1A, O2, O4, O6A, O8, O15, O16, O18A, O25B, and O75.

16. An O-EPA bioconjugate composition obtained by the process according to claim 5.
